(19) United States
(12) Patent Application Publication
(10) Pub. No.: US 2020/0064172 A1
(43) Pub. Date: Feb. 27, 2020

(54) Wireless Device for Measuring Gas and Fluid to and from a Patient

(71) Applicant: Teliox LLC, Miami, FL (US)

(72) Inventors: Piotr H. Tabaczewski, Miami, FL (US); Henry T. Zakkour, Miami, FL (US)

(73) Assignee: Teliox LLC, Miami, FL (US)

(21) Appl. No.: 16/550,251

(22) Filed: Aug. 25, 2019

Related U.S. Application Data
(60) Provisional application No. 62/722,038, filed on Aug. 23, 2018.

(51) Int. Cl. G01F 15/06; A61B 5/00; A61B 5/20; A61M 5/168; A61M 16/10; A61B 5/087; A61B 8/04; A61B 8/12; A61B 10/00

(52) U.S. Cl. CPC G01F 15/063; A61B 5/0002; A61B 5/208; A61M 5/16886; A61M 16/1005; A61M 2016/003; A61B 8/04; A61B 8/12; A61B 5/746; A61B 10/007; A61B 5/201; A61B 5/087

(57) ABSTRACT

Devices and systems compare incoming and outgoing gas and fluid flowrate measurements with each other and with additional physiological measurements of a patient to trigger an alarm to healthcare providers of an issue with a patient. The device can include a sensor that measures incoming flow of gas and fluid and a sensor for measuring the outgoing flow of gas and fluid (for example, urine) from the patient. The sensors can be connected to wireless transmitters to send data describing the flow to a computer processor. The computer processor receives the data from the processors and generates an alarm based on comparisons between the input flow and the output flow. The devices and system can be used to detect issues in a patient by monitoring a flow of intravenous fluids being administered to an amount of urine being generated. The devices and system can be used to detect issues in a patient by comparing a flow of gas being administered to a patient compared to blood gas content in the patient.

### CROSS-REFERENCE TO RELATED APPLICATIONS

This application claims the benefit of U.S. Provisional Application No. 62/722,038, filed Aug. 30, 2018, which is hereby incorporated by reference.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The invention relates to devices for collecting medical data from multiple sensors. In particular, the invention relates to devices for monitoring patients that include a flowmeter in combination with an additional sensor.

### Description of the Related Art

It is accepted that people who become so sick that they must be hospitalized need to be closely monitored during hospitalization and after discharge because they are at high risk of fatal events. The need of close monitoring extends well into recovery period, at least thirty (30) days, and possibly longer after discharge from the hospital, for acute medical conditions and even in patients with full clinical resolution. Patients with chronic conditions are at risk of worsening and ideally should be monitored constantly. To provide the best care, it is very important to detect evolving fatal events and worsening of chronic conditions as early as possible. The timing is extremely important. The time from normal to serious or even fatal condition for cardiovascular and respiratory problems can be measured in a few minutes, cerebrovascular in minutes to hours, renal in close to one hour to several hours, and metabolic in general somewhat longer. To intervene in timely fashion, “fast” saves life and money, prevents suffering related to disabilities, decreases hospitalizations, and decreases readmission rates. To intervene, it is important to detect problems not only as early as possible but also as accurately as possible. False positive alarms increase costs.

Historically, healthcare providers have responded to and relied on patients' complaints: e.g. not feeling well, fever, chest pain, and shortness of breath. The complaints are subjective and, therefore, not very reliable. Diagnoses based on the complaints result in many false positives and delayed reporting. Objective measurements of key health status parameters, which are disease specific, have been introduced. Examples include vital signs, oxygen saturation, and capillary blood glucose level. The collection and evaluation of real-time data at time intervals or continuously, is called “monitoring”. Monitoring can be done at the measurement site or remotely. When done remotely, monitoring is called telemonitoring. The significance of monitoring is to measure objectively, in real time, key body functions, with an aim to detect changes as early as possible in order to allow practitioners to intervene in timely fashion. The significance of telemetry is to evaluate monitored values remotely.

The field of health monitoring is being developed rapidly and is directed toward consumers or to use in the hospitals. Despite rapid improvements, there are several limitations of current monitoring systems. The focus of monitoring is towards measurement of bodily status, which can be described as “output” parameters. During their daily lives, people perform physical activities, their health condition changes, and they take medications. These stimuli can be described as “input” parameters.

The theory of correlating between input/output parameters when done in scientific fashion by an individual skilled in art (e.g. a physician) is known to provide powerful insight into the health status of monitored individuals. The correlation of general physical activity is sometimes called, in lay terms, “performance analysis”. In consumer markets, performance analysis is widely available, but not in a rigorous way that is needed in medical evaluations. In professional medical applications, rigorous medical-level evaluations that generally are categorized as “stress tests” are cumbersome, available only in institutions. The results of these medical-level tests are difficult to interpret, particularly by unskilled persons, and, therefore, provide significant financial and intellectual barrier for the consumers.

Strikingly, there is a paucity of input/output correlation in medical analysis. Examples of unavailable relational data include absence of data relating oxygen consumption to oxygen saturation, blood pressure level to urine production, and administration of fluids to urine production. Even when the previously listed data is individually available, that data is not related. This lack of correlation is especially striking as it is a backbone of scientific analysis and the foundation of science. Accordingly, a need exists to put medical methods on par with scientific methods in medical management.

Evaluation of Urine Production

A theoretical application of correlating data relates to evaluation of a condition or renal function, which relies on measurement of two key parameters: volume of urine production and estimated glomerular filtration rate. Both values are critically important. Volume of urine produced depends on glomerular filtration rate and reabsorption rate of urine and is an early and best predictor of developing renal failure, septic shock, response to treatments of fluid congestion, and dehydration. Glomerular filtration on the other hand rate is important for monitoring kidney status and injury, both chronic and acute, dosing medications, and estimation of kidney function. The following discrete urine tests exists. The evaluation of urine production can be performed by observing the volume of urine collected. An alternative for measuring urine production utilize fluid flowmeters. Existing fluid flowmeters for urine communicate data via a wired connection to a dedicated device for recording the signal being output by the urine flowmeter. These wired urine flowmeters are problematic with ambulatory patients. As a separate device, spectrophotometers can estimate GFR and turbidity of urine. A separate pH meter is used to measure urine pH. In a separate device, the estimation of urine conductance can be done with conductometer. There are no comprehensive real time analyzers of urine that would measure: urine GFR, turbidity (absorbance) with spectrophotometry, pH, and conductance.

Evaluation of urine production rate is notoriously inaccurate because urination volume is reported by nursing staff based on discrete readings of urine collected from bags, measuring cylinders, and patient reporting, which requires patient cooperation. Healthcare providers use collection hats to measure urination. A collection hat is a collection container, preferably made of plastic, that is graduated for measuring collected urine volume by visual observation. Data collected at home is even less trustworthy as there is no reliable outpatient system that would measure objectively real-time urine production. The estimation of glomerular filtration rate is done routinely using creatinine levels in serum. The problem is that this laboratory value is frequently not accurate in acute settings. In acute kidney injury (AKI), many days can be required for new steady levels of creatinine that is used routinely for this purpose, which results in underestimating severity of kidney injury. Other factors including reduction in muscle mass, liver injury, and dietary changes, among others, all of which make this test less accurate. As a result, the serum concentration of potentially toxic medications that need to be dose-adjusted based on GFR sometimes may reach toxic or sub-therapeutic levels.

In summary, the evaluation of urine production is very poor, especially in the sickest patients, at night or at home. There are no input/output response protocols as response to diuretics, fluid administration, and blood pressure. Because the estimation of urine output is so poor, the estimation of patients' fluid balance is poor as well. The process is not only inaccurate but slow as well and, because of this, the adequate interventions are delayed, kidney injuries are missed, and patients' discharge to home is delayed. There is no reliable and practical system that would estimate live glomerular filtration rate. There is no comprehensive, portable non-invasive urine analyzer that would measure physical urine parameters in real time fashion.

Liquid Flow Sensor

Liquid flow sensors continuously measure the flow rate Q=(∂V/∂t) of liquids passed therethrough and output a signal that is a function of the measured flow rate. A liquid flowmeter sold under the trademark SENSIRION LD20 is sold for the purpose of measuring drug delivery precisely and in real time and further to be applied in biomedical devices, advanced infusion therapy, wearable drug delivery devices, urine catheters, surgical instruments, and bioreactors. The SENSIRION LD20 continuously performs measurement of ˜0.5 ms length. The output flowrate value corresponds the average of all individual 0.5 ms measurements xᵢ, since the last read out. The digital calibrated liquid flow signal read from the SENSIRION LD20 sensor is a 16-bit signed integer number (two's complement number ranging from −32768 . . . 32767. Note that with the sensor's output limit being ±1300 ml/h, it will only output values in the range −26000 . . . 26000). The integer value can be converted to the physical value by dividing it by the scale factor (liquid flow in ml/h=sensor output÷scale factor).

Mass Flowmeter

Mass flowmeters continuously measure the flow rate Q=(∂V/∂t) of gases passed therethrough, generate a signal that is a function of the measured flow rate to an output. Gas flowmeters sold under the trademarks SENSIRION® SFM3300 and SFM3200 are sold for measuring bidirectional flow volumes, which are standardized for temperature and pressure, and are fully calibrated for air, N₂, and O₂ gases. The sensors also feature medical cones for pneumatic connection to the respiratory cycle and have mechanical interfaces for a user-friendly electrical connection.

Discrete Flow Sensor Systems

Various liquid and gas flow sensors exist. These sensors are used in discrete systems. A discrete system uses the data from the flow sensor and displays a result. A discrete system is “discrete” because the system does not correlate the result with results generated by another, different sensor, and with results generated by another, different flow sensor. In the discrete system, the sensor might be connected to a dedicated computer. However, the computer only works with data from a particular flow sensor and not data from additional sensors, particularly additional flow sensors.

Manual Analysis of Multiple Sensor Data

Even though the significance to patient health of comparing input data to output data is known, healthcare providers have failed to implement the comparison in patient treatment. Healthcare providers do not implement such comparisons because correlating two sets of data from two discrete sensor devices requires a level of expertise that requires knowledge typically only possessed by physicians and that also requires too much calculation, analysis, and time to allow the physician to perform the physician's other responsibilities. Configuring a respective alarm for each discrete system will fail to detect some problems that are discernable only with at least both sensors. And, without an automatic alarm, a diagnosis becomes dependent on periodic data collection and correlation, which leads to delays in detection that could be detected by continuous, automatic monitoring and alarms.

Devices for conducting real-time spectrophotometry of urine or other body fluid are not presently available.

### SUMMARY OF THE INVENTION

An object of the invention is to provide a device for correlating input/output data with medically useful accuracy and reliability. To achieve this object, the device includes an input sensor whose data is recorded as a function of time and an output sensor whose data is recorded as a function of time, and whose data is correlated to each other by that time. The device then generates a signal, for example an alarm, based on an evaluation of the input data and the output data (or based on data is derived from the input data and/or output data).

The device according to the invention, which bases decisions on correlated input and output data, has been found to enhance patient safety with earlier detection than evaluations based on input data or output data alone. The recording of input data as a function of time and output data as a function of time along with the time information increases the information in a patient's medical history data, and this correlated data can be studied and used for historical and predictive analyses. The device and the recorded correlated input and output data can be used to calculate risks and benefits of treatment. Because the device can generate a signal based on the continuous analysis of input data and output data, the device can send the signal to healthcare providers or to patients immediately without a delay of hand calculating decisions at discrete moments, e.g. when a patient urinates or when a provider visits the patient. Finally, a single signal is a form that providers and patients can understand easily.

The live, wireless, portable, automatic, direct measurement of body fluids flow including urine is currently not available. This invention allows early detection developing renal failure, urinary catheter malfunctions, intravenous delivery malfunctions, monitoring flow in other medical drains such as a thoracentesis tube or a Jackson-Pratt (JP) drain.

A further object of the invention is to provide a wearable device for transmitting flow rate data to a server via a wireless protocol. The wearable device includes a flow rate sensor with an output. The flow rate sensor generates a signal that is a function of flow rate and sends the signal to the output. A wire connects the output of the flow rate sensor to a wearable computer. The computer encrypts the signal to generate an encrypted signal. The computer has a wireless access point that can connect to a wireless access point on a network. The encrypted signal is transmitted to a server connected to the network.

A further object of the invention is to provide a device and system for detecting issues in a patient by monitoring basic physiological functions, gas and fluid input, and gas and fluid output, heart rate, arterial blood and central venous pressures, urine qualities, and temperature, and for alarming a patient or healthcare provider based on a continuous comparison of at least two of the basic physiological function.

A further object of the invention is to provide a system for continuously monitoring patients for renal failure in an order of minutes after its onset by comparing a volume of fluid being input into a patient compared to a volume of urine being output by a patient. More particularly, the invention calls for a device that calculates the volume of liquid being input into a patient by integrating a continuously measured flow rate that is measured over a period. Likewise, the invention calls for a device that calculates the volume of urine being output by integrating a continuously measured flow rate over a period. The device can include a first liquid flowmeter for measuring the flow rate of fluids being input into the patient (i.e. an “input liquid flowmeter”) and a second liquid flowmeter for measuring the flow rate of urine being output by the patient (i.e. an “output liquid flowmeter”), and a computer connected to the liquid flowmeters for recording time and the flow rates at the time.

A further object of the invention is to provide a system for continuously monitoring patients for catheter malfunction within minutes of the malfunction. The system includes the device described in the previous paragraph. In addition, the computer (or an additional computer connected to the computer) includes an alarm. The computer activates the alarm when the calculated volume of urine being output falls below a threshold that is below the calculated fluid input, where the output volume being compared is for a period ending after the end of the period of the input volume. The lag in time can be the amount of time the patient that a healthcare provider expects the patient to require to excrete and filter the input fluid into urine.

With the foregoing and other objects in view, there is provided, in accordance with the invention, a sensor system including multiple sensors, that can be functionally described, and input and output sensors connected to a computer. Where the computer correlates the data from the input sensor and the output sensor by time. The device can transmit an alarm by comparing data derived from the input senor and data derived from the output sensor.

The system can be used for diagnosing a patient's health by comparing amounts of ingoing and outgoing fluids in the patient. The first sensor in this example of the system can be a first fluid flow sensor, which also can be referred to as an output fluid flow sensor. The fluid flow sensor directly measures a rate of urine production and continuously outputs a computer readable signal that is a function of the rate of urine production in real time. The computer receives the signal from the fluid flow sensor. The second sensor can be a second fluid flow sensor, which also can be referred to as an input fluid flow sensor. The second fluid flow sensor directly and continuously measures a rate of intravenous (IV) fluid administration. The second fluid flow sensor outputs a second computer-readable signal that is a function of the rate of IV fluid administration. The computer integrates the first signal over a period and generates a volume of urine production over that time. Likewise, the computer integrates the second signal over the period and generates a volume of fluid administered over the time. The computer can detect changes in flow rate by calculating a time differential of the flow rate (i.e. ∂Q/∂t).

The device can transmit a warning signal to indicate potential renal failure when the volume of urine production over the time drops a threshold amount below the volume of fluid production over the time. The threshold amount will be adjustable according to level of the individual patient.

The device can transmit a warning signal to indicate a potential malfunction in a urinary catheter (e.g. clogging and kinking) by detecting an absence in urine production, particularly after detecting an input of IV fluids.

Early detection of renal failure and catheter malfunction allow for earlier intervention, correction, and treatment.

An object of the invention is to transmit wirelessly flow rate data from a flow rate sensor to a computer. In accordance with this object, a device includes a flow rate sensor with an output that is connected by wire to a computer. The computer includes a wireless transmitter. The flow rate meter generates a signal that is a function of flow rate and transmits the signal to the computer. The computer derives a wireless signal according to a wireless protocol that is a function of the flow rate and that includes time detected per time. The wireless signal is passed to the wireless transmitter, which transmits the signal for reception by a remote wireless receiver. The device including the sensor and computer can be portable by having a size and weight that can be carried and that includes its own power source (e.g. batteries).

An object of the invention is to provide a device that is portable. A portable device is more likely to be used by a patient, particularly when outside a hospital. In addition, a portable device is less likely to be disconnected by a patient, particularly when moving to the bathroom to urinate.

An object of the invention is to provide a device that is automatic. An “automatic device” is one monitors itself and sends a signal to a healthcare provide or caretaker when a value exceeds a preset threshold. In contrast, a “manual device” is one that outputs results that must be observed by a provider or patient to detect when a value exceeds a preset threshold.

A further object of the invention is a device and system for real time analysis of urine. As an output sensor, the system can include a pH probe to measure the pH of urine being produced as a function of time. The pH can be associated with the data of the input sensor, which is also generated as a function of time. An example of an additional output sensor is a spectrophotometer. The spectrophotometer will measure light absorbance spectra of urine as a function of time. The spectra can then be correlated to the data from the input sensor, which is also a function of time.

A further object of the invention is to provide a system that includes an input fluid flowmeter and output flowmeter equipped with pH probe and digital spectrophotometer. The goal of the invention is to measure and correlate acidification or alkalization of urine in response acidifying or alkalizing infusion. Another goal of invention is to monitor the presence of spectrally active agents in urine. These can be endogenous body products like urobilinogen or exogenous substances like methylene blue or methotrexate. A further goal is to measure presence of electrical charges (i.e. ions) in urine as a function of infusion of fluids, for example, normal saline.

In accordance with the objects of the invention, an integrated portable system is provided that includes a measuring unit, a computational unit, a data transmission unit, and a communication unit capable of monitoring and analyzing and transmitting body physiological parameters. An integrated portable system according to the invention can meet the following objects.
The measuring unit can measure real-time cardiac, respiratory, and renal function.
The computational unit (i.e. computer) can analyze and cross-reference measured values.
The data transmission unit can send computed data to users with security clearance to monitor basic physiological functions, warn of worsening and alert of danger.
The communication unit can communicate data to different users according to level of clearance to allow collaboration and facilitate deep understanding of significance of data.
The system can be customizable at all levels.
The system can be portable.
The data can be recorded for subsequent observation and analysis.
The data transfer and communication will be wireless and medical level encrypted.
The invention further relates to devices for cross-referencing data by vertical statistical analysis (i.e. analysis of current sensor outputs and comparison to the stored information), and horizontal statistical analysis (i.e. comparing sensor readings to the stored data from different users who use the same system.

The cross-referencing is understood as a relation of sensor readings to each other. The sensor readings can be the same sensor reading at or over different times or to other sensor data at the same time or at different (longer, shorter, lagging) times.

“Vertical statistical analysis” is understood as statistical comparison of sensor data, sensor specific or cross-referenced over time of the same subject. This capability allows to estimate dose effect of pharmacological treatments and interventions with statistical powers. Horizontal analysis is understood as finding similarities of sensor reading patterns between different subjects. Furthermore, the software serves as a communication tool of sensor data to user, family, designated individuals, organizations, physicians, hospitals etc. If a user gives permission then the sensor output (either raw or analyzed, cross-referenced, and/or compiled) can be transmitted for research and commercial purposes. The computer is programed to have analytical capabilities to assess the medical urgency status and monitor fluctuations with focus on baseline deviation: in particular, to measure patient worsening. The deterioration can be graded to urgency levels.

The invention is also related to sending automatically the sensor data and patient location to medical emergency services as well as to designated neighborhood healthcare providers. The computer can be programmed to send a service request that includes the location of the patient. The request can be generated automatically when sensor data is needed. The request also can be initiated manually initiated by a user to transmit to designated providers.

The invention includes a portable device having a liquid or gas flow rate meter connected to a wireless transmitter for wirelessly transmitting the flow rate data to a remote computer or server. The device can be incorporated as part of a larger system.

In accordance with the objects, the invention includes software for performing the following functions:
operating the sensors;
providing calculations;
providing a warning system;
providing a location of the patient; and
providing encrypted and safe real time multiuser communication tool to see raw, analyzed data (correlative) and transferable results regardless of patient and provider location.

When receiving urine from a collection source (e.g. a catheter), the fluid flow sensor measures urine production on spontaneous urination. If no urination is recorded over a given time interval, the concern for urinary retention will be raised.

Combining Sensors

Sensors alone are very important, but they provide one dimensional output. To combine several together and relate to each other creates added value. A system that evaluates data from the following sensors can be used to distinguish and identify issues: a heart rate monitor, a blood pressure sensor, a device for measuring urine output. For example, when dehydrated, the heart rate is high, blood pressure tends to be low, and urine production low. When you give fluids, then the heart rate decreases, blood pressure increases and urine production increases. Throughout, oxygen consumption is unchanged.

When a patient has congestive heart failure, heart rate can be high, blood pressure low and urine output low. When fluids are administered, then the heart rate will go even higher and blood pressure response may be variable: unchanged, or lower, and urine production will not increase, and oxygen to saturation ratio will worsen.

Diseases have distinctive patterns of physiological parameters, sepsis, atrial fibrillation, pneumonia, and COPD. As it is difficult to diagnose the disease at first moment, the response to treatment is crucial to confirm primary problem. The relation of responses: If A goes up and B goes down and C and D is unchanged, they most likely diagnosis is E.

Additionally, heart rate, peripheral oxygen levels, peripheral blood pressure, temperature are measured in the same manner. However, comparable single sensor systems for these parameters are currently on the market.

Portable, Real-Life, Wireless, Automated Multi-Sensor System for Monitoring Basic Physiological Functions in the Acute-Care Setting and Home

The system is particularly applicable to two major areas. First, systems for detecting worsening multiorgan vital functions. Second, systems for monitoring and warning caregivers of worsening multiorgan vital functions.

The system provides the ability to monitor multiorgan vital functions in real time, to detect worsening multi-organ functions earlier, to send warning message to caregivers, and to guide treatment. The system is intended to operate both in the hospital and home or any location that has cellphone or internet reception, which is practically everywhere. The system offers superior monitoring capabilities to allow hospital level monitoring to home patients with cardiac, respiratory complaints or others. The system allows healthcare providers to treat patients at home who otherwise would have to be in the hospital because of poor baseline discharge safety and readmission risk concerns.

In Institutions

The system can alert healthcare providers who are not primarily monitoring a given patient when measurements being monitored by the system fall outside selected parameters. As stated previously, most healthcare providers are responsible for numerous patients (i.e. a set of secondary patients). At any given moment, the healthcare provider may be reviewing one of the secondary patients, who then can be referred to as a primary patient for the duration of the focused monitoring. The system provides alerts to healthcare providers about secondary patients even when they are focused on a different primary patient. After being alarmed by the system, the healthcare provider can focus on the patient generating the alarm and reassess and recalibrate treatment to stabilize that patient, even before of completion of early diagnosis workup (e.g. CT or Echocardiogram).

The multi-sensor system measures changes of vital functions within a second of measuring. The system provides this fast measurement by correlating multiple sensor output continuous in real time. The sensor allows a healthcare provider to see these correlative data remotely. Examples of how the system alarms can alarm healthcare providers quickly to speed changes in patient treatment protocol as follows: measure vitals including more comprehensive set (blood pressure (“BP”), CO₂, possibly CVP), initiate stabilizing measures (minutes), reassess (thirty (30) minutes to three (3) hours), and make changes, diagnose (one to three hours), make changes on the fly (every three (3) hours). If too busy to adjust and there is worsening of physiological parameters, then the system sends an alarm message.

At Home

Usually, patients are kept in the hospital initially for diagnosis and later for safety. With the system, it will be enough to keep patients, especially those with respiratory problems, in the hospital only for a diagnostic period (one to three days) and shift remaining treatment to outpatient locations (e.g. home, nursing homes). By shifting the remaining treatment to outpatient locations, the system provides a new concept of treatment of people. This new concept of treatment moves people from the risks of hospital and moves them to safer, patient preferred outpatient locations. The system monitors patients in outpatient locations closely as well as when traveling, and connecting to home doctors; in most cases, closer than patients who are being treated in hospital but without the system. The improvement will be recorded with analyzing trends. The patient centered monitoring system in which sensors can be added or discontinued based on new needs will make this system special and very important tool to use in patient home centered care.

The Wireless Transcutaneous Capnography Sensor

Currently, a correlative, wireless warning system of ventilatory failure is not existing. Patients with risks of retaining of carbon dioxide (CO₂ can lose consciousness because of rise of blood carbon dioxide in the presence of normal oxygen level. The problem of carbon dioxide intoxication is that it develops insidiously, and the increase of carbon dioxide level is not felt until too late. Patients with neurodegenerative conditions or muscle dystrophies as ALS, Duchenne, terminal COPD are good examples. These people can die even when family is next room. Because of this, patients and their families live with constant concern for safety of their loved ones. There is great need of wireless monitoring system for these people. This invention provides wireless monitoring and warning system for patient and family.

The Wireless Oxygen Flowmeter Sensor in Combination with Wireless Transcutaneous Capnography and Oxygen Saturation Sensors

Currently, there is no device that correlates use of oxygen (oxygen flow) and at the same time carbon dioxide (CO₂) and oxygen level. Too much oxygen or too little is bad. Currently, oxygen delivery is an “open loop” system, which means it is not automatic. Patients regulate their oxygen delivery based on perception of shortness of breath and are helped by pulse oximeters. However, the perception of shortness of breath correlates poorly with actual blood oxygen level. It is common for people to feel okay even with dangerously low blood oxygenation or feeling short of breath with good oxygen level. At night there is no regulation of oxygen delivery at all if the patient is asleep. The solution for these problems is a closed loop; an automatic oxygen delivery device whereby the oxygen is delivered to the desired setting. In this way, it would be possible to achieve two goals: 1. provide oxygen to objective needs, and 2. to be able to measure how much oxygen is needed for given blood oxygen saturation level (with a flowmeter). Currently, there are non-FDA approved devices on the market that deliver oxygen to specific target of oxygen level (closed loop delivery) but without measurement of carbon dioxide. Closed loop oxygen delivery without measurement of carbon dioxide level can be very dangerous. The reason is that when people are comfortable because of getting enough oxygen they are at risk of slowing breathing and retaining carbon dioxide. Retention of carbon dioxide will lead to confusion and loss of consciousness and potentially death. The addition of a carbon dioxide safety measurement makes close loop oxygen delivery safe. It will improve substantially performance of the system by delivering oxygen not only to blood oxygen saturation but also to the combination of blood oxygen and carbon dioxide levels. In addition, skin reading of oxygen is more accurate than traditional pulse oximetry. All taken together, the tri sensor system allows safe closed-loop, output-targeted oxygen delivery.

With closed loop oxygen delivery, it is possible to establish a standardized respiratory performance test: evaluation of home oxygen needs for patient discharge. This test, usually done on discharge day is very important and done by respiratory therapists. It is important for determining if a patient is ready to be discharged to the patient's home and for determining if a patient need home oxygen. Commonly, respiratory therapists perform the test by walking patients and assessing blood desaturations and respiratory symptoms. When symptoms of shortness of breath occur, the oxygen level is increased, and oxygen need is established. The whole test lasts about one hour and frequently is not done in time that will result in home discharge delays.

Closed loop oxygen delivery systems can be also used to test if patients with respiratory problems can fly safely. This dose response test is done in specialized respiratory labs by allowing patients to breathe oxygen at various concentrations to see if they can safely fly. However, it would be much better to perform the test at the airport just before boarding. The wireless close loop able to do performance test wirelessly and transmit data remotely to trained examiner would solve the problem.

The wireless, portable system with a close loop oxygen delivery will measure how much oxygen is needed for the patient to keep safe levels of blood oxygen saturation and carbon dioxide levels. The portable system can be fully automated; thus, it is not therapist dependent. The test can be performed by a nurse or physical therapist. This way there will be no delay to discharge patient home. Because it is standardized, patient will be able to repeat the same test home and this way measure health status.

Wireless Oxygen Flowmeter Sensors Combined with Transcutaneous Capnography, Oxygen Saturation, Fluid Flow Sensors, and Heartrate Sensors

The invention provides for a comprehensive system that combines a wireless oxygen flowmeter sensor with a wireless transcutaneous capnography sensor, an oxygen saturation sensor, a fluid flow sensor, a heart rate sensor, a blood-pressure sensor, a central venous pressure sensor, and a temperature probe.

In the hospital, the comprehensive system will allow early assessment of therapy. At home, the comprehensive system will provide comprehensive monitoring. The comprehensive system will provide early alerts of deterioration in the patient's condition.

Evaluation of Glomerular Filtration Rate (GFR), Urine PH, Conductance, Urinary Bleeding.

The evaluation of GFR is of very important in medicine because it is a principal marker of kidney injury. Another reason that it is very important is that all medications that are eliminated by kidney (most of them) need to be dosed using this parameter. As there are no good and practical methods to measure real time GFR using filtration rate, a surrogate method is used. Most commonly, GFR is calculated from serum level of creatinine at steady state. Steady state concentration is a result of balance between past and current creatinine production and elimination rate and therefore GFR calculated this way is a combination of historical and present GFR value with unknown contribution of each other. In conditions in which GFR changes rapidly as in acute medical emergencies or with acute renal injury, the new GFR is frequently very different from recent past and which makes this method not reliable. In addition, intrinsically, creatinine-based calculations of GFR are affected by many factors that influence creatinine metabolism as: muscle mass, age, medications, diet and many others. Inulin clearance test is considered the gold standard method of GFR estimation. The reason that inulin (plant derived carbohydrate) is so reliable is that is not endogenously produced or metabolized and is eliminated exclusively by kidneys and without being reabsorbed. Over time, good alternatives have been found (sinistrin, dextrose, and radiological contrast). The “classic” GFR calculation method relies on the injection of inulin and plotting serum concentrations to urine concentrations over time. Later, this method has been validated with other compounds as well. Less time-consuming protocols have been developed over time: with less frequent urine and serum measurements, using reporter agents. Reporter agents are compounds that are marked with an entity that allows better detection (radiolabeling, fluorescence) so that the concentration of the compound can be calculated with measurement of signal derived using label signal intensity, measured with appropriate detection methods (Geiger counter, spectrophotometry). However, all available methods rely on multiple measurements of concentration of chemical compound or reporter agent in serum, thus needing repeated and timed blood draws making this approach not practical in everyday practice

Another common problem in medicine is bleeding from urinary tract. The most common causes of bleeding into urine occurs after surgeries, in urinary tract infections, and after treatment with anticoagulants. Sometimes bleeding can be life threatening and early detection and estimation of blood losses would be very important. In clinical practice, it is very difficult to estimate the rate of urinary bleeding or assess changes as even small, non-life-threatening bleeding colors urine very intensely red, which is frightening for patient and concerning for medical staff.

The range of urine pH is large. The large range is a result of the kidney's function to regulate the acid/base balance in the body by eliminating excess acid or base. Therefore, urine pH can be considered an output signal. Urine pH is important in patients with kidney stones, renal acidosis. Urine pH needs to be maintained at certain pH when eliminating toxic medications. It is done by infusion of acid or base and measuring urine pH at time interval by laboratory test.

Urine conductance is dependent on the presence of ions. It also undergoes changes during the day depending on ions presence diet, for example salt. Measurements of urine electrolytes is important in several common conditions: dehydration, syndrome of inappropriate diuresis syndrome, disturbance in sodium levels. Currently real time monitoring of urine conductance is not done.

Urine turbidity (absorption of light) is an important marker of infection, dehydration. Monitoring of urine turbidity is not done in clinical practice.

As many conditions result in disease specific pattern of combination of spectrometry, pH, conductance, turbidity, the cross-referencing of these parameters to each other over time would assist significantly in early diagnosis of many medical conditions, monitoring changes and clinical responses there is need for such a device. Recent advances in miniaturization, increase of sensitivity makes it possible to build e multisensory device that would be very useful in this area. Near infrared spectroscopy could be used to detect carbohydrate reporters in urine (inulin, sinistrin, and dextran). Detection of fluorescently labeled reporter molecules should be possible with spectroscopy or fluoroscopy. With known urine flow rate and compound concentration, the elimination rate can be measured and real time GFR can be calculated basing solely on urine testing. In theory, the volume of distribution (the volume into which the injected compound will be diluted into) would be needed for GFR calculation. This value can be estimated with available formulas and no measurement may not be needed. For most accurate value, single blood test may be needed to measure serum concentration of injected compound. On the other hand, in order to estimate changes of GFR in the same patient, no blood urine draws should be needed as GFR changes can be calculated by comparison of elimination rate. Thus, the utility of this method will depend solely on urine detection of compounds or reporter agents in the urine with spectrophotometry or fluorometry. For example, if the compound/reported will be eliminated twice faster, then GFR will be twice higher. Urinary bleeding can be measured by the presence of signal intensity of characteristic red emission of hemoglobin in visible light. The concentration of urine electrolytes and the value of urine pH can be measured with miniaturized conductometers and pH meters respectively Therefore, accurate real time changes of GFR, urinary blood loss, detection and monitoring of infection, dehydration, monitoring of liver function could be done with urine analysis with simple, non-invasive device. Urine pH is important in some cased but because, in itself, it is not considered and important parameter alone to monitor, monitoring of urine pH is not done routinely, and monitoring of urine pH has not been evaluated in clinical practice. Similar considerations are valid in analysis of other bodily fluids and analysis of fluid composition in other areas (environmental).

Evaluation of Oxygen Needs

Currently, all patients who come with symptoms of shortness of breath or who are at risk of developing it are being treated with oxygen. Many especially admitted with chest pain should not use oxygen at all as there are no clear benefits of hyperoxia and it may be harmful. The response to oxygen saturation is measured with pulse oximetry, in most patients, with spot checks. In sick patients there is telemonitoring available. Here is how it is typically done: candidate patient will be treated with supplementary oxygen at initial dose of choice, for most two liters per minute (2 l/min) and this flow rate will be titrated up or down until comfortable and/or a goal (i.e. setpoint) of oxygen saturation is reached. Feeling of shortness of breath is poorly correlated with blood oxygen levels. The dosage adjustments are not accurate as the oxygen flowmeter is not digital, that results with significant flow rate error of 1.75-2.25 l/min for 2 l/min rate. Later, the oxygen dose is adjusted sporadically when a patient complains of shortness of breath or hypoxia is noted. When a patient is feeling good and not hypoxic, the oxygen dose stays unchanged. The process of decreasing oxygen delivery rate is much slower as there is certain comfort feeling that patient is safe with high dose of oxygen. As a result, almost anyone who is hospitalized would be initially given oxygen as a preventive and safety measure despite of the fact that when oxygen is overdosed it can have harmful effects. See Shuvy et al. This approach is also costly (˜$10/day). As a result, knowing the patient's oxygen needs at discharge is difficult. For patients who have been receiving oxygen throughout the stay but who are not short of breath, at the discharge oxygen is simply discontinued. For patients who are short of breath and/or with documented hypoxia during hospitalization on the last day the need of home oxygen is evaluated with home oxygen evaluation test. The test is done by a respiratory therapist, who choose random time to do it and walk with patient for few minutes and documenting oxygen saturation levels and records oxygen dose that is needed to achieve target value and relieve feeling of shortness of breath. This test is highly performer dependent. When there is established need of home oxygen for discharge, the patient will be set up with home oxygen agency. At home, the responsibility of oxygen dosage is traditionally on the patient alone and there is no supervision or close monitoring of needs.

In summary, the prior-art process is inaccurate and not standardized. It is done arbitrarily on the last day of hospitalization without reference to past performance. It is time consuming. There is lack of follow up for patients at home that would correlate oxygen consumption with oxygen saturation, especially done with standardized protocol. Carbon dioxide levels are not used as target parameters for oxygen delivery for most patients who receive oxygen. At discharge, the estimation of home oxygen use is poor. There is no continuity of real time assessment of oxygen/carbon dioxide levels to oxygen consumption rate. The common practice is to overtreat patients with oxygen. This is associated with harmful side-effects and higher costs.

Wireless Oxygen Flow Sensor and Devices Equipped with Wireless Oxygen Flow Sensor.

In accordance with the objects of the invention, a wireless, wearable, real-time oxygen flowmeter addresses and solves significant problems associated with oxygen therapies. The device includes an upstream (relative to the sensor) tube to be connected to an oxygen source. The gas is flowed via the upstream tube (also referred to as the “in tube”) and runs through a mass flowmeter and continues to a patient via a downstream tube (which is also referred to an “out tube”). Therefore, functionally, the device can be considered to have an “in” sensor. The oxygen flow data is collected for any oxygen or gas delivery line equipped with the flow sensor. An output of the flow sensor is connected to an input of a digital computer. The digital computer also may be referred to as a computer, logic unit, or box. A wire is a particularly reliable device for interconnecting the flow sensor and the digital computer. The digital computer can be sized to be wearable. The digital computer is programmed to collect data received from the flow sensor. In particular, the flow sensor records flow rate data as a function of time from the sensor. The digital computer can encrypt the collected data and generate a signal carrying the encrypted data. The signal complies with a wireless communication standard and is transmitted by an antenna. The antenna can be an integrated part of the digital computer or as a separated antenna connected to an output of the digital computer. The digital computer can provide power to sensors connected to the digital computer. The transmitted signal is sent wirelessly to a wireless access point that is connected to a server. The wireless access point converts the wireless signal into encrypted data and sends the encrypted data to a server. The server can decrypt the encrypted data and can store the data. The stored data can be analyzed by the server or sent to another computer for analysis. The device can include additional gas flow rate sensors, where each gas flow rate sensor is connected to a respective gas source. For example, a gas flow sensor can be connected to a nitrogen source in order to measure the flow rate of nitrogen being supplied to a patient. A downstream tube from each gas flowmeter sensor can be connected to each other and a single supply cannula can be used to deliver a combined gas. Examples of additional gasses that can be supplied and monitored with a respective gas flow rate sensor include carbon dioxide, carbon monoxide, and helium. The digital computer can be reusable and able to withstand medical sterilization.

In accordance with the objects of the invention, a device is provided in which the gas “input” flow sensor can be coupled with an “output” sensor. Such a device can be referred to as an “input/output device” or “input/output system”. Output sensors include not only output liquid and gas flow sensors, but also sensors that measure an effect on a patient that is related to the input. For example, output sensors include oxygen saturation probes, dissolved oxygen concentration, and carbon dioxide levels. The output sensor can include a probe for measuring a value (i.e. an effect) and generates a signal that is a function of the measured value and transmits the signal to a digital computer, which passes the data to a server in a way that is similar to the oxygen flowmeter, which was described previously in this application. When both a signal from an input sensor and a signal from an output sensor are received, then, in addition to the previous functions, a computer reading data from the server can be programmed to analyze a dose/response relationship. For example, what concentration of oxygen in a supply flow is needed to achieve a given oxygen saturation or carbon dioxide level in the patient. Another type of relationship would be oxygen saturation to carbon dioxide depending on oxygen flow rate.

Evaluation of Carbon Dioxide Levels

Evaluation of a patient's carbon dioxide level is very important in patients receiving oxygen and in patients who are not able to breathe normally. Currently, carbon-dioxide level measurement is performed bedside with non-portable devices that measure the carbon dioxide concentration from exhaled air (i.e. direct-stream or side-stream capnography) or directly by gas transmission through the skin (i.e. transcutaneous capnography).

Wireless Capnography Sensor

The transcutaneous capnography sensor is an existing type of probe. The probe is attached to a lightweight portable digital computer that can collect data and transmit the data wirelessly. Prior-art flow sensors include a large box and a probe attached by wire to the box. Transcutaneous capnography sensors serve to measure blood level of carbon dioxide and oxygen via tissue transfer. Capnography sensors also can measure a patient's heart rate. The action of the sensor is different from traditional pulse oximetry sensors, which measure oxygen content with spectrophotometry. The transcutaneous capnography probe is more accurate in people who are very sick. The accumulation of carbon dioxide is the most sensitive method to detect ventilatory failure (inadequate breathing). Inadequate breathing can be very dangerous and lead to death. People at risk include patients suffering from Chronic Obstructive Pulmonary Disease (COPD), Amyotrophic Lateral Sclerosis (ALS), muscle dystrophy, and opiate overdoses. The device can be equipped with a wireless alert system that provide remote monitoring and safety warnings. The ability to correlate oxygen level and carbon dioxide level is crucial to understand respiratory status. This is not possible with pulse oximetry only. The device collects data from input and output sensors in a form that can be correlated, for example by time, and transmits the data to a server. A digital computer connected to the server can be programed to evaluate respiratory status in people in their natural home environment, evaluate daily patterns to detect worsening, optimize treatments, and check if current respiratory treatment result in better performance. The device can include a capnography sensor connected to a tube carrying exhaled air to measure the carbon-dioxide level. In both these cases, additional output sensors can be added to the device for measuring output parameters such as respiratory rate, spirometry, and tidal volume.

Design and Features of Receiving, Transient Data Storage and Transmission Unit (Box)

A wearable, telemonitoring system includes a data receiving unit (i.e. a digital computer) from sensors. Depending on its purpose, the device can have additional features like advanced analytical capabilities or data storage.

Digital Computer for Receiving and Transmitting Transient Sensor Data

The invention includes a wearable digital computer (i.e. a “box”) capable of collecting data from multiple sensors design and that can connect to any needed sensor that can be chosen from a larger set of possible sensors. The digital computer includes an input that can be connected to a lead (i.e. wire) from a sensor. The input is connected to a logic board that receives signals from the sensor and is programmed to encrypt the data extracted from the signal and to send them as a signal wirelessly to a server. The data transmission can be also integrated with power/charging unit, capable to supply power for at least one hour of operation. For longer operation, an optional external battery will be used. Also, the unit can be supplied with power from outlets. It will have also several features of artificial intelligence: when receiving normal baseline reading, it will operate in sleep mode, making measurements at longer time intervals. When one of sensors would record abnormal reading then the device will make more frequent measurements. In the case that one parameter is at baseline and the other abnormal, then data collection frequency will be skewed towards abnormal parameter.

Design and Features of Operating Software.

The data from sensors via box will be transferred to password protected and encrypted server. An analytical system includes a computer for analyzing data from the various sensors. These new analytical features are possible because of the design of the multi-sensor system. The collected and stored information, both raw and analyzed, will be owned by the patient. Data will be stored in at least two physical locations, will be readily accessible and sent wirelessly upon request. Analytical evaluations will be done at server level: recording of tracing, calculations, correlations with statistical powers, extrapolations. The operating software will have real time communication capabilities to establish audio and visual contact, customizable alarm/warning adjustment, location (GPS) capabilities, notification (patient, physicians, families etc.). The changes of alarm settings will be done at administrator level and upon request by provider or user. There will be three versions of software: hospital level, physician level and patient level. These systems will have the data prepared in a way that would serve a purpose of different level of needs and understanding.

The software works as follows. For simplicity, only data from liquid flow, gas flow, and pulse oximeter will be used but it should be noted that the same principles would apply to all multi-sensor systems. In case of more sensors, more advanced and complicated analytical system will be available. Healthcare providers will be able to see all patients in their care, visually represented as a list or tiles (supplemented with identifiers), on displays of the computer, pad or any portable device. This first screen will have information identifying patient. All patients ever monitored will be seen on administrator level, with active users prioritized. Provider (doctor, nurse etc.) will be able to see only patients with established relationship. Individual user will be able to see only own data. The identifiers will be constantly updated. When the monitoring device indicates a concerning value (i.e. an alarm), the color of the identifier will change from blue to yellow and shifted up to the list with time stamp on the identifier. When panic tracing is detected, then the identifier will change to red with optional sound alarm. When an identifier is double clicked, the patient data will be accessed for review. When double clicked, the time stamp will be deleted but the color will stay until the value normalizes, but the patient's position will drop behind non-accessed identifiers in its own category (concern or panic). By selecting the patient of interest, the next screen will feature all tracing in the form of overlaying histograms with possibility of adding or removing individual graph. Each histogram will represent a value over time. On this screen, it will be possible to view the data over variable time interval. The next screen will show correlative data, if one set of data would correlate with each other. A subsequent screen will contain “patient stats”: amount of urine in a day, oxygen use in a day, average saturation, etc. Another screen will feature setting control tools. Control tools will include alarm setting, choice of correlation analysis, choice of statistical values. For a given patient, either a preselected set of settings or a correlative and statistical alarm can be used or can be customized for individual patient. For illustration, if the warning value for urine production will be urine production of less than thirty milliliters per hour (<30 ml/hr) or panic alarm of ten millimeters per minute (10 ml/min) then two values will be entered in alarm settings: numerical (10 or 30 ml) and time duration. In this case, the average will be calculated on the server level at preselected “marching forward” time intervals, for example, ten (10) minutes. When a panic value is measured, then the appropriate alarm signal is sent. The progression of urine production will be seen on next screen and correlated to any other parameter on following, for example correlation of urine production to oxygen saturation. The correlation will have statistical value with confidence level. Two types of correlations will be done: qualitative (variance analysis; if urine production correlates with oxygen saturation) and quantitative (T student; average value oxygen saturation to change of oxygen delivery rate). The correlations can be compared to historical data and to expected (physiological) responses. The system will be operating life and will have ability to perform statistics on ongoing basis. The patient stats will be in the form of value: amount of urine production in a day, amount of oxygen use in a day, average oxygen saturation and so on. The software will allow live communication as well as of sending prerecorded information in form of video, audio or text. The recorded tracing will be stored and will be owned by users who can use it as they see it fit. Based on needs, performance reports, analysis of long-term trends will be sent to providers or patients in a customizable and understandable fashion in order to evaluate progression of disease, correlation with treatments and status of worsening/recovery.

Comprehensive System

Overall structure of comprehensive system the same except it will be equipped with multiple sensors. The possible sensors are: fluid flowmeters in and out, urine spectrophotometry, conductance and PH, gas flowmeters (oxygen, nitrogen, gas mixtures), oxygen, carbon dioxide level, respiratory rate, spirometry, arterial blood pressure, central venous blood pressure, heart rate, cerebral perfusion, electroencephalogram (EEG), temperature (Earbud, Tempdrop, Oura Ring, Ava), capillary blood glucose levels (Libre, G5 Mobile, Enlite, Alcon).

Overall, the size of the comprehensive system is no greater than the size of a current telemetry system that measures only heart rhythm but is capable to monitor non-invasively all vital functions and other optional parameters. Currently, to measure vital functions, routinely every six (6) hours, patients are awoken, which interrupts their sleep/wake cycle and contributes to more delirium and less satisfaction. Having the automated system in place would allow to eliminate staffing costs related to measurement especially at night. Also, by having alarms sent wirelessly to nursing station would eliminate sound alarms located currently at bed site as in infusion pump. Most importantly, such a system would provide higher safety with less intrusiveness and staffing reduction.

According to the invention, a comprehensive system connects and endows existing sensors with new capabilities. The comprehensive system equips existing hardware with these sensors to creating a new sensor, which is a single piece of hardware that binds together the components to create a system with new abilities.

The comprehensive system is portable, wireless, automated (i.e. does not require human monitoring), more reliable, and faster at generating results.

The comprehensive system not only connects existing devices; it employs sensors that have not been applied as used in the comprehensive device.

Ultimately, the comprehensive system will improve patient safety by preventing and shortening hospital stays and will provide to patients more control over their life. Finally, the comprehensive system will make medicine more cost effective and precise. It will allow to quantify benefits and provide estimation of confidence levels.

The invention improves the capabilities over the prior art by utilizing several sensors to generate correlative analysis. In addition, the system according to the invention is fully customizable: choice of sensors, choice of correlations, customizable thresholds, choice of recipients of alarms and correlation data, accessibility to software for further analysis, and applicability to statistical analysis of dose response data for a given individual.

Measurement of Urine Production with Devices via Fluid Flowmeter

In congested patients, good monitoring of urine production is very important for patients with fluid congestion and fluid depletion. The cornerstone or treatment of fluid congestion is administration of diuretics and monitoring fluid balance. It is known that response to diuretics is of critical value to patient recovery the principal therapeutic goal is maintaining negative fluid balance. Despite of this, in clinical practice even at best institutions and in very controlled settings of clinical trials, it is difficult to obtain reliable data on fluid intake and diuresis (Testani et al.). In addition, the response to therapy is variable in different individuals. The failure to assess progress of diuresis in the hospital and outpatient is leading costs of hospitalization and readmissions (Parinello et al.). The wireless digital system will allow automated and accurate estimation of dose response to diuretics and establishment of protocolized management based on dose-response relationship. Patient-specific dose response profile (baseline profile) will be established and when hospitalized next time patient can start with appropriate management from the very beginning. This will make faster to find appropriate and safe dose, establish dose that will work at home. The portable system will allow to monitor diuresis in the very critical post discharge period in order to intervene and prevent readmission. In addition, by having accurate value of urination, much better assessment of fluid balance can be done.

In dehydrated patients, the term of “dehydration” will be used here in broad sense as low intravascular volume. Dehydration can happen when not having adequate drinking, losing fluids due to diarrhea, bleeding or dilating of vessels as a part of infection (e.g. sepsis). Poor perfusion can lead to ischemia (confusion, stroke, MI). Kidneys are a “sentinel organ of poor perfusion” and the first sign is making less urine. In addition, when poor blood perfusion is extended over time, then temporary or permanent kidney injury can occur resulting in costly and prolonged hospitalization. For example, in cardiac patients undergoing elective cardiac surgery, thirty percent of patients develop acute kidney injury. See Hansen et al. Even in low risk elective orthopedic surgeries, the injury rate is high: depending on the study, from 2.2% (Perregard et al.) to 10% or even higher. For this reason, it is of critical importance to detect decrease of urination rate as timely correction can result in injury prevention. The automated monitoring system capable detecting the earliest sign of pending renal injury would especially at night when monitoring is poor would address this problem.

Measurement of Urine Function with Comprehensive Analyzer Consisting of Flowmeter, Spectrophotometer, PH Meter and Conductometer

In order to measure an accurate filtration capacity of kidneys, one can inject a substance that can be detected by spectrophotometer. The system capable to accurately measure filtration rate would consist of fluid intake flowmeter that can measure time and volume of injected agent and urine wireless spectrophotometer together with urine flowmeter. Inulin (which is considered the “gold standard”) and few other substances are eliminated exclusively by kidneys making the man ideal marker of glomerular filtration rate. The problem with it is that it can be only detected with chemical analysis. Other methods used in the past as radiolabeling are not practical. Interestingly, fluorescently labeled compounds have similar urinary clearance profiles as not labelled (for example inulin FITC or Dextran Texas red) when tested with classical blood assays (Wang et al.). Combination of a urine flowmeter (volume) and spectrophotometer or fluorometer (concentration) would allow to measure elimination of these compounds via urine and thus measure accurately real time GFR. The clearance time of the substance calculated based on geometry of the clearance curve (time to maximum, time to clearing 30, 70%, shape of the curve). The method should also work possibly with other compounds (methylene blue, also natural dyes as derived from red beets carrots etc.). Inulin, sinistrin, and dextran are likely to be preferable candidates as these compounds have been studied thoroughly and are safe to administer in humans.

In certain instances, it is important to change and monitor the pH of a patient's urine. It is done to eliminate toxic agents. It is important that urine pH is if certain value. One good example is treatment with methotrexate. During this chemotherapy, the urine should have pH above eight (>8). It is done by injecting with bicarbonate. Currently, the urine pH is measured by collecting urine sample and sending to laboratory every six (6) hours. Based on the value, the bicarbonate rate is adjusted. With continued measurement of urine pH and bicarbonate flow rate with wireless urine sensor and a fluid intake flowmeter sensor, it is possible to adjust urine pH automatically and optimally, eliminating human errors and laboratory tests, while maintain full control of the process. Similar technique could be used with overdoses of tricyclic antidepressants, salicylates, phenobarbital, and herbicides.

When urinary bleeding appears, it is difficult to assess the significant the bleeding. Using spectrophotometry to measure the intensity of red color and knowing urine volume, the level of bleeding can be easily calculated. In addition, a trend (i.e., increasing or decreasing) of the bleeding can be calculated.

By combining measurement of several properties of urine (e.g. pH, spectrophotometry, absorbance, and conductivity), comprehensive urine monitoring can be done to provide early detection of infection (turbidity), acute tubular necrosis (decrease of urine production and turbidity), and renal tubular acidosis (decrease of pH). In addition, conductance could be (Kavukcu et al.) combined with other parameters, such as urine flow and pH, in monitoring treatment of SIADH, renal injury. Conductance alone is not very useful alone and, therefore, is currently not being used.

An imbalance between fluid administration and urine production provides a very sensitive first warning of impending renal failure or injury. In practice, the imbalance is frequently missed because human monitors require time in the amounts of hours to discern differences in the incoming and outgoing fluids. Issues with measurement accuracy are caused by tests being administered by different healthcare providers, untracked changes to IV bags, and untracked emptying of urine bags.

The issues in human detection are demonstrated by the ongoing need for faster detection in institutions such as hospitals and SNFs.

The system allows automated recording of all IV injections (e.g. morphine) for safety of patients. In contrast to prior-art nursing manual recording, this feature will allow to record all IV interventions including antibiotics and chemotherapy.

Measurement of Oxygen Use with Gas Flowmeter

Oxygen is used routinely in the hospitals in all patients with shortness of breath and in patients at risks despite oxygen being known to be harmful if not needed (Demiselle at al.). Traditionally, a supply of oxygen is administered and adjusted arbitrarily, without strict protocols, and not necessarily in accordance with a patient's needs.

Currently, in the hospital or at home, oxygen flow is regulated manually. The flow rate is displayed on an analog meter, a healthcare worker periodically looks at the meter and then logs the discrete meter reading in a log. There is no continuous recording or constant monitoring of the oxygen flow except in ventilated patients. It is obvious that it would be important to know how much oxygen patients receive, when, with what device. In addition, it is important to know by how much and when the oxygen flow rate is changed. If at home, patients use oxygen, how much, when, and from what device should be tracked. The wireless oxygen flowmeter solves this important problem. The wireless flowmeter can be inserted in any oxygen delivery line, regardless of device that delivers oxygen, send it wirelessly, provide warning if sudden flow stops, display use pattern and calculate oxygen use per unit of time.

Patients who are ventilated are different because ventilators are complicated devices and have oxygen flowmeters built in. At the same time when ventilated patients have oxygen probes on. Both values are recorded. It is not done in non-ventilated patients because flow rates are not recorded from oxygen lines.

Wireless Oxygen Flowmeter Sensor

Currently no portable wireless oxygen flowmeter is available.

The invention includes a wireless oxygen flowmeter sensor. This sensor generates a computer-readable oxygen-sensor signal that is a function of a patient's oxygen intake. The sensor is attached to a lightweight portable computer that is capable collecting data and transmit the data wirelessly, which is a function of the oxygen sensor signal. The sensor measure oxygen flow directly. The oxygen sensor system is portable, weighs less than one gram (<1 g), and is configured to be carried by a patient. The oxygen-sensor measures oxygen flow, regardless of source. Sources of oxygen can be pressurized tanks, oxygen concentrator, noninvasive preaching devices like a CPAP and BiPAP, and ventilators. Sensors, each with a unique ID, can be inserted into lines from specific sources and then the system can determine when and how much oxygen is used. The oxygen sensor can broadcast a wireless signal upon detecting an oxygen delivery malfunction, which is detected when the oxygen flow falls below an alarm threshold. The sensor can calculate changes of oxygen delivery rates from the continuous measures of flow rates and broadcast a warning signal when a change in flow rate exceeds a given threshold, measure oxygen daily consumption and usage patterns. These features of this device make it ideal to monitor oxygen use patterns. The use patterns at home are important piece of information for the doctor and insurance. This information so far was provided by the patients mostly from memory. Patients also use different devices, portable tanks, concentrator, noninvasive devices. The sensor inserted in line with device ID will solve this important problem of accurate recording: how much oxygen, when using what. It can be used by insurance companies, doctors and oxygen delivery service to serve patient better and save costs. As the portable wireless oxygen flowmeter is a mass flowmeter, similar calculation, after gas-specific adjustments can be made for other gases (air, nitrogen, carbon dioxide or others)

Combined Wireless Oxygen Flow Sensor with Pulse Oximeter

The combination of oxygen flowmeter and oxygen saturation creates input-output system, whereby the oxygen delivery rate can be related in dose response fashion to blood oxygen saturation. Any mismatch or unexpected changes of oxygen flow rate to oxygen saturation level can be rapidly detected, investigated and acted upon. In addition, changes of the ration of oxygen dose to oxygen saturation can be correlated with medical interventions. For example, when medications are given with goal to improve symptoms of shortness of breath it can be evaluated if treatment resulted in objective findings of better utilization of oxygen (less oxygen use for the same oxygen saturation). The statistical analysis of responses after repeated use, will create a baseline respiratory treatment response profile for the patient. The response profile can be compared with dose changes, and to therapeutic responses with other treatments. When feeling sick, the deviation from the baseline respiratory performance profile can be detected early, estimated in severity and interventions can be initiated that can result in prevention of further deterioration. This may be called “preventive” respiratory intervention with goal to abort of development of vicious cycle and ultimately to prevent hospital admission or readmission. Currently this type approach is not used at home as there is lack of appropriate device that could be used in ambulatory setting. In the simplest form, the respiratory baseline can be created by averaging of oxygen saturation to amount of oxygen used. As oxygen use is very dependent on exercise, the test should be done in very standardized setting: sitting in a chair or on treadmill with preset exercise effort level. More advanced system would include commercially available close loop oxygen delivery system (DIMA, Free Oxygen). Close loop oxygen delivery will deliver oxygen to the preset oxygen saturation level. In this case, the respiratory baseline can be created by averaging an amount of oxygen over time that results in maintaining certain level of oxygen saturation. To increase the dose-response measurement range, to lower saturation values, nitrogen gas can be added. This may be needed as many patients may not need high oxygen levels at the rest. In this case, the patient will breathe a defined mix of oxygen and nitrogen via nasal cannula. Nitrogen flow will be given at steady rate and oxygen will be delivered with close loop oxygen delivery system in order to achieve preset oxygen saturation. Flow rates of both gases will be measured accurately with digital flowmeter. The oxygen utilization test will be performed till stable baseline will be reached.

Combined Wireless Oxygen Flow Sensor with Pulse Oximeter and Capnographer

Providing a system that correlates oxygen dose to oxygen saturation and carbon dioxide level would allow for comprehensive evaluation of respiratory function. Similarly, as in the previously described method, oxygen flow rate can be measured directly from a gas line regardless of the delivery modality (e.g. nasal cannula, mask, CPAP, BiPAP). Oxygen saturation can be measured from the skin, finger, or ear lobe with any appropriate probe. The capnography sensor can be transcutaneous, side-stream and direct-stream. The addition of a capnographer is a significant improvement that not only adds useful new set of data but also new level of safety. As it is more complicated system, it will be used selectively in people who have abnormal carbon dioxide levels or in whom carbon dioxide level needs to be closely controlled. One group are patients in whom respiratory effort is driven by hypoxia (e.g. COPD exacerbation), as soon as hypoxia is corrected with supplementary oxygen, the respiratory drive will slow and result in accumulation of carbon dioxide. If elevation of carbon dioxide is not corrected confusion and ultimately carbon dioxide narcosis can occur. Another group are the patients with defect of ventilator effort, at high risk of carbon dioxide retention (muscular weakness as in ALS, muscular dystrophy, obesity hypoventilation syndrome, depression of ventilation by opiates and other drugs). Patients with acute stroke, who receive oxygen, tend to hyperventilate which will cause hypocapnia that would lead to constriction of cerebral vessels (Iscoe et al.). In these patients, is important therefore to monitor and adjust carbon dioxide level closely with goal of permissive hypercapnia. Therefore, it can be said that maintenance of adequate oxygen saturation is most importance but in certain group of patients it is not only parameter that needs to be closely monitored. The best method would be to deliver oxygen with close loop oxygen delivery with appropriate target value that according to the etiology can be in broad range (85-95%). In general, this target value range corresponds to about change of about 5 L supplemental oxygen via nasal cannula. As oxygen delivery allows rather broad delivery range, there is a room to for modification in order to target carbon dioxide levels: downward in exacerbation of COPD and hypoventilation, safely upward in stroke patients. With side stream capnography, it is also possible to measure respiratory rate, spirometry, tidal volume. All these parameters could be included in correlative algorithms.

A pulmonary performance test is a test done occasionally at a specialized lab. The pulmonary performance test measures many respiratory functions. Typically, a pulmonary performance test is performed once every few years. The pulmonary performance test provides a great evaluation of lung function. However, the study is done once only at the state of health that you are in.

With the system according to invention, a patient's pulmonary function will be measured many times, maybe even daily, which will provide average values with a standard deviation. The measurement by the system may not be as good as “real” pulmonary performance test, but, because the system performs a test so many times, the baseline will be more accurate, and deviation of the baseline will be assessed better. A gas flowmeter can be incorporated into a breathing mask or through a mouthpiece, to measure gases (e.g. oxygen) being consumed by a patient. In addition to sensor for measuring oxygen in/pulse oximetry and capnography out, the system can include sensor for measuring spirometry, tidal volumes, and respiratory rate values. The data can be correlated with the other data being recorded.

Examples of Use of Wireless, Wearable Systems that Include Gas Flowmeter, Liquid Flowmeter (Simple and Comprehensive), Wireless Capnographer.

Wireless gas flowmeters, liquid flowmeters, and urine spectrophotometers can be integrated with already existing wearable sensors: pulse oximeters, temperature probe, blood pressure, ECG, EEG, brain perfusion. Other non-wearable sensors (e.g. skin capnographers) can be made wearable. For the sickest patients, a central arterial probe would be useful. In certain patients, portable central venous probe would be needed as well. These sophisticated systems would make big difference in patient care. The multisensor system with variable, medical problem targeted specific set would make big difference in the way medicine is practiced. The possible most illustrative applications are presented below but the list is much longer. In addition to described use examples, the device can be used in any condition that would need telemonitoring and cross-referencing of multiple parameters: responses to medical treatments (blood pressure to blood pressure pills, heart rate to arrhythmia medications, airway obstruction to bronchodilators, combining of albumin or thiazides with loop diuretics on diuresis, salt infusion to urine conductance to mention just few; evaluations of base performance (long term monitoring of chronic conditions as pulmonary fibrosis, pulmonary hypertension, lung and heart transplants, COPD, congestive heart failure, advanced chronic kidney disease and others); early detection of adverse events: monitoring of physiological function during clinical trials in order to detect side effects, carbon dioxide intoxication in hypoventilation patients

Specific Application: Post-Surgical Patients

Post-surgical patients are especially at risk of failure of the following body functions: renal failure because of fluid shifts, cardiovascular and multi-organ failure because of sepsis, ventilatory failure because of anesthesia medication and fluid shifts, and respiratory failure because of aspiration risks, pulmonary embolus and heart injury because of inflammation among most common. Posts-surgical patients are frequently treated with pain medications, are weak and confused thus not able to communicate when in distress, especially at night when there is also less nursing attention. Currently, no automatic, correlative, portable, wireless comprehensive monitoring system with adjustable parameters is available to detect early acute problems. A system capable of real time monitoring: heart rate, blood pressure, temperature, oxygen saturation, carbon dioxide level, respiratory rate, urine production with wireless data transmission and capable to transmit warning message would be capable not only to send alert of important medical issues but help to diagnose the nature of medical problem. For example, if fluid shifts would cause intravascular depletion, then the first sign would be increased heart rate followed by drop blood pressure and decrease of urination. Respiratory parameters and temperature would not be changed. On the other hand, if vascular congestion with pulmonary edema would happen, then primary cardiac (heart rate) respiratory parameters would be affected (respiratory rate, decrease oxygen saturation, increase of oxygen use), followed later by decrease of urination. In case of pneumonia there will be also fever and if pneumonia result in sepsis, respiratory decline will be flowed by drop of blood pressure and urine production. Therefore, the monitoring of capnography, pulse oximetry, heart rate, temperature, blood pressure and fluid balance would increase patient safety to the next level by early detection, followed by intervention and later followed to evaluation if the intervention was adequate. The process can be partially or fully automatic and can be easily standardized thus providing adequate backup of human personal monitoring and computational limitations.

Other Common Conditions

The multi system is customizable and addition of specialized probes gives flexibility to create patient centered monitoring system. For example, by adding a brain perfusion probe one can focus on early detection of stroke. In addition, an EEG probe can be added to detect seizures.

As above, the system monitors the relationship of data from one to the one other. In addition, a change of one to relation to another. Decisions (i.e. alarms) can be activated by comparisons of data regarding breathing in conjunction with data regarding patient temperature and hypoxia (vis-à-vis measured blood oxygen content). For example, if a physician suspects pneumonia, then the patient is checked for fever, hypoxia, and low CO₂, and breath rate. The system can provide a comparison of patient temperature (i.e. decreased fever following administration of administered acetaminophen) and volume of oxygen input to correct hypoxia and breath rate to diagnose the patient as having pneumonia. However, (give oxygen), then carbon dioxide will normalize (will breathe normally), but if it is not happening then you must explore different hypothesis like pulmonary embolus.

In other words, in medicine, the most important is relation of one thing to another. And the most important is timing. Therefore, the system is constantly relating one parameter to another in relation to intervention and transmitting warnings.

Specific Application: Early Diagnosis and Establishment of Appropriate Management

Many patients carry multiple chronic conditions, commonly: congestive heart failure (CHF), asthma, chronic obstructive pulmonary disease (COPD), renal disease, heart disease, and diabetes in variable states of severity. Patients are at different ages. When a new condition, for example pneumonia, occurs frequently chronic conditions worsen as well. It is common that a patient is diagnosed at the same time with acute pneumonia, acute or chronic CHF exacerbation, and acute COPD exacerbation. After an immediate diagnosis, healthcare providers treat the patient with potent medications. Only after observing a patient after a few days of treatment can a healthcare provider render a better diagnosis, based on which, the healthcare provider may discontinue some of the medications. It is important to note that frequently these treatments are synergistic, neutral, and even counteracting. In acute medical problems, there is also an emphasis to correct and maintain physiological functions and electrolytes (vitals+blood chemistry) within physiological ranges regardless of the cause.

A high performance input/output system according to the invention provides a method to evaluate treatment effects, stabilize vital functions, and electrolytes as quickly as possible. With choice of potent and short acting medications with defined physiological responses, it is possible to perform several attempts of “management trials” in the span of a few (preferably, three (3)) hours, starting in ED or some even at the time of arrival of emergency services provided. The examples of such a fast intervention follow: fluid bolus, oxygen, norepinephrine, esmolol, nitroprusside, albuterol, nitroglycerine, furosemide, and dobutamine. As these agents can be grouped in exactly opposite pairs: fluid bolus/furosemide, esmolol/dobutamine the opposite effect is expected. Because on their quick action, even if an initial approach is not good, a higher dose can be administered subsequently because of the short acting duration. With higher doses, the measurable responses will be more dramatic allowing for high confidence and possibility of establishing correct management at earliest time. Similarly, with correct estimation of urine output, and concentration of important electrolytes in both urine and serum (potassium, magnesium), correct supplementation protocol can be initiated. With a system for appropriate and high fidelity monitoring, a fully automated process can be provided, in which patient's physiological functions will be maintained in desired ranges (blood pressure, heart rate, fluid balance, oxygen saturation, carbon dioxide levels) with fully automated system, using initially short acting medications, later to be transitioned to maintenance longer acting: e.g. esmolol to metoprolol, nitroprusside to amlodipine. Similar process can be used with electrolyte correction. The ability to verify treatment response as early as possible will result not only in better outcomes but in reaching faster and more accurate prognostic decisions: in emergency departments to admit or discharge home; once admitted to assess discharge readiness faster, at home to try to manage, go to primary/specialist, call an ambulance. This system will be very useful in monitoring of patients discharged to rural communities. Patients with chronic conditions can use a multi-sensor system customized to their needs at home to create of baseline and monitor status of the disease. At this stage, longitudinal data analysis will be performed (progression of the disease, seasonal changes, and evaluation of treatment responses). If feeling worse, patients would compare test results to their previously established baselines. If the patient is stable and not too far of baseline, then the patient should try to use medications and see if improving while continuing monitoring. If worse and not improving, then patient may contact physician and follow instructions. If the patient is still not successful, is feeling much worse, or is deviating from his or her baseline (danger or panic levels), then the patient should call an ambulance. If the patient is too sick and unable to communicate, then the system will make an automatic call with global position satellite (GPS) location and initiate communication protocol (audio, visual or both). The monitoring will continue into ED, during hospitalization, and after discharge, documenting status changes and response to treatments. This historical data of the event will be analyzed and will assist in management of future exacerbations, monitoring returning to baseline and detecting new worsening. Similar scenarios will be used in patients with a new disease (i.e. previously healthy patients). In this case, a baseline will not be available. The monitoring levels, choice of sensors, and measured parameters will be customized. The monitoring can be done remotely and the data sent locally and/or “home base providers” who are familiar with patient, providing this way portability and at the same time continuity of care.

What is needed is a comprehensive monitoring system that is tailored to patient's needs, that will allow to assess quickly effect of treatment and estimate improvement rate. The device should be wearable to be used both in the hospitals and at home.

For example, when a patient is admitted to a hospital, a healthcare provider will begin treatment based on available information and accepted clinical best clinical judgement. Because the treatment is based on assumptions and a yet-to-be completed test results, the initial treatment being prescribed is often not correct. Typically, the healthcare provider reassesses the patient and the treatment daily, for example, as a physician conducts daily rounds in a hospital. As a result, the patient would be receiving improper treatment until the healthcare provider reassess the patient, which can be up to twenty-four hours later. Any delay in diagnosing errors in prescribed treatment could prevent the patient from obtaining the proper treatment and could expose the patient to injuries being caused by giving the wrong treatment. Extending the delay only increases revision of initial treatment after so many hours when not correct can cause life threatening delays of starting better management.

After patient admission or after detecting a worsening of a patient, standard hospital procedure is to measure a patient's fundamental parameters of physiological functions (i.e. vitals) and to initiate stabilizing measures within minutes of admission or detection of a critically worsening condition. Then, after one to three hours, procedures call for the healthcare provider to re-diagnose the patient. Then, after twelve hours, the healthcare provider reassesses the patient, except when emergency occurs. As a result, newly arising problems and slowly worsening problems (i.e. wrong path) are not noticed until twelve hours later.

Fundamental physiological parameters of physiological functions should be measured and assessed as accurately and timely as possible and, if changed, the information should be sent immediately to make adjustments. It can be deduced from the art presented here that such a system would be capable of make these adjustments automatically in order to reach “ideal parameters for given patient.” It is here understood that “ideal parameters” would be given by trained physician. If baseline is known, then ideal parameters would be best baseline. If baseline is unknown, then general baseline can be defined based on general ruled and current medical condition. The order in which the patient's baseline parameters are corrected: heart rate first or blood pressure first will be decided by physician but is expected that the process can be partially or even fully automated.

The reason for urgency and accuracy is that, at the beginning of treatment, it is difficult to predict who would worsen and if chosen parameters are correct. Similarly, close monitoring is important for post procedure patients.

For Cross-Referencing

In the prior art, cross-referencing requires use of collection of simultaneous data from multiple sensors these systems do not exist because lack of number of sensors. Because of lack of portable wireless systems and lack of cross-referencing, there is no integrated portable system consisting of measuring unit, computational unit, data transmission unit, communication unit capable of monitoring, analyzing and transmitting body physiological parameters.

Veterinarial Applications

The devices, systems, and methods, which are described in this application, are not only applicable to human patients; they also can be applied to animal patients in a veterinarial setting.

Other features that are considered as characteristic for the invention are set forth in the appended claims.

Although the invention is illustrated and described herein as embodied in a device and system for detecting issues in a patient by monitoring gas and fluid input and gas and fluid output, the invention should not be limited to the details shown in those embodiments because various modifications and structural changes may be made without departing from the spirit of the invention while remaining within the scope and range of equivalents of the claims.

The construction and method of operation of the invention and additional objects and advantages of the invention is best understood from the following description of specific embodiments when read in connection with the accompanying drawings.

### BRIEF DESCRIPTION OF THE SEVERAL VIEWS OF THE DRAWING

FIG. 1 is a schematic view of a first embodiment of a system according to the invention, the system including a wireless flowmeter device with an input fluid flow sensor for monitoring intravenous fluids being delivered to a patient.

FIG. 2A is a schematic view of a second embodiment of the system according to the invention, the system including a wireless flowmeter device with an indwelling catheter and an output fluid flow sensor for measuring urine output.

FIG. 2B is a diagrammatic, sectional, partial view of the second embodiment of the system of FIG. 2A being shown inserted in a female patient.

FIG. 2C is a diagrammatic, sectional partial view of the second embodiment of the system of FIG. 2A being shown inserted in a male patient.

FIG. 2D is a diagrammatic, sectional partial view of the second embodiment of the system of FIG. 2A being shown inserted in a female patient.

FIG. 3A is a schematic view of a third embodiment of the system according to the invention, the system including a wireless flowmeter device with an external catheter for males and an output fluid flow sensor.

FIG. 3B is a schematic view of a third embodiment of the system according to the invention, the system including a wireless flowmeter device with an external catheter for females and an output fluid flow sensor.

FIG. 3C is a diagrammatic view of the third embodiment for males shown in FIG. 3A.

FIG. 3D is a diagrammatic view of the third embodiment for females shown in FIG. 3B.

FIG. 4A is a partial sectional view of a fourth embodiment of the system according to the invention, the system including a wireless flowmeter device with an internal catheter for males and an output fluid flow sensor and with the internal catheter shown inserted in a penis of a patient.

FIG. 4B is a partial section view of the system shown in FIG. 4A with the catheter withdrawn from the penis of the patient.

FIG. 5A is a schematic view of a fifth embodiment of the system according to the invention, the system including a wireless flowmeter device with a urine collection hat and an output fluid flow sensor.

FIG. 5B is a schematic view of the fifth embodiment of the system, which is shown in FIG. 5A.

FIG. 6A is a diagrammatic top view of a toilet bowl sheet shown with the toilet bowl sheet fully deployed on a top surface of a toilet bowl rim.

FIG. 6B is a diagrammatic top view of a toilet bowl sheet shown with the toilet bowl sheet fully deployed on a top surface of a toilet bowl rim.

FIG. 6C is a diagrammatic top view of the toilet bowl shown in FIG. 6B with the toilet bowl sheet removed.

FIG. 6D is a diagrammatic top view of the toilet bowl sheet shown in FIG. 6A connected to a wireless device for measuring urine flow.

FIG. 6E is a diagrammatic top view of the toilet bow sheet shown in FIG. 6B connected to a wireless device for measuring urine flow.

FIG. 6F is a diagrammatic top view of a second embodiment of a toilet bowl sheet covering only a front portion of the toilet bowl.

FIG. 7A is a diagrammatic top view of a toilet bowl sheet with the toilet sheet fully deployed on a top surface of a toilet bowl rim with the seat lifted.

FIG. 7B is a front sectional view of the toilet bowl sheet shown in FIG. 7A while connected to a wireless device for measuring urine flow and with the toilet seat lowered.

FIG. 7C is a top view of the toilet bowl shown in FIG. 7A with the toilet sheet is folded back on a top surface of a toilet bowl rim with the seat lifted.

FIG. 7D is a front sectional view of the toilet bowl sheet shown in FIG. 7C while connected to a wireless device for measuring urine flow and with the toilet seat lowered.

FIG. 8A is a top view of a plate for collecting urine seated within a toilet seat.

FIG. 8B is a front section view of the plate shown in FIG. 8A.

FIG. 9 is a schematic partial view of a wireless flowmeter device according to the invention.

FIG. 10 is a schematic view of a closed loop system for regulating oxygen flow to a patient using a wireless flowmeter device with an input mass flowmeter and an output mass flowmeter.

FIG. 11 is a schematic view of an open loop system for analyzing urine including a wireless flowmeter device with an output fluid flow sensor.

FIG. 12 is a schematic view of a closed loop system for regulating oxygen flow to a patient using an input mass flowmeter, an output mass flowmeter, and additional sensors.

FIG. 13 is a schematic view of a wireless noninvasive combined central venous pressure meter and arterial pressure meter.

FIG. 14 is a diagrammatic view of a central blood vessel sensor used in the wireless noninvasive combined central venous pressure meter and arterial pressure meter shown in FIG. 13.

FIG. 15 is a schematic view of a closed loop system for analyzing gas input and gas output and fluid input and urine output of a patient.

FIG. 16 is a schematic view of an open loop system for regulating oxygen flow from a backup source to a patient.

FIG. 17A is a diagrammatic plan view of a winged infusion set with a spectrophotometer shown in an open position.

FIG. 17B is a diagrammatic plan view of the winged infusion set with the spectrophotometer shown in a closed position.

FIG. 17C is a diagrammatic perspective view of the spectrophotometer shown in FIGS. 17A and 17B, in an open position.

### DETAILED DESCRIPTION OF THE INVENTION

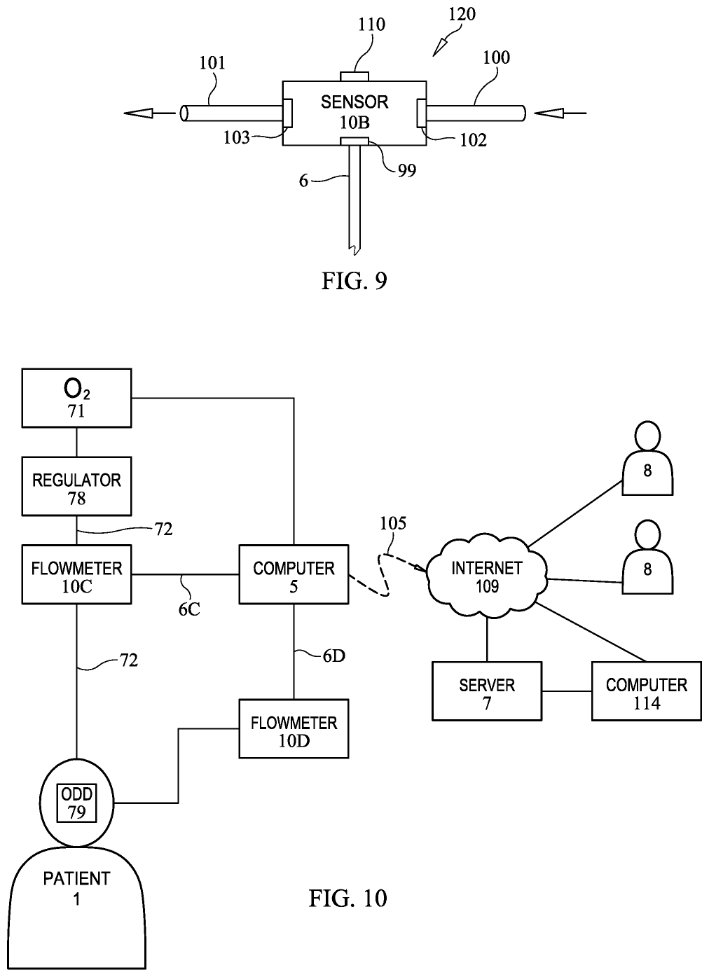

FIG. 9 shows a preferred embodiment of a wireless flowmeter device 120. Input cannula 100 carries a liquid or gas to an input connector 102 of the fluid flow sensor or mass flowmeter 10. Output cannula 101 connects to an outgoing connector 103 of the fluid flow sensor or mass flowmeter 10 and carries liquid or gas from the fluid flow sensor or mass flowmeter 10. The fluid flow sensor or mass flowmeter 10 generates a signal that is a function of flow rate of liquid or gas through the fluid flow sensor or mass flowmeter 10. A lead 6 is connected to an output 99 of the fluid flow sensor or mass flowmeter 10. The lead 6 carries the signal from the fluid flow sensor or mass flowmeter 10 to a digital computer, which is not shown in FIG. 9. The signal is encrypted by the digital computer into an encrypted signal. The digital computer encodes the encrypted signal according to a wireless protocol into a wireless signal. The digital computer includes a wireless transmitter that broadcasts the wireless signal. A wireless access point receives the wireless signal and decodes the wireless signal into the encrypted signal. The wireless access point is connected to a network. A server is connected to the network. The wireless access point transmits the encrypted signal to the server via the network. The lead 6 further carries electricity for powering the fluid flow sensor or mass flowmeter 10. For measuring a liquid flow rate, a preferred embodiment of the fluid flow sensor 10 is sold by SENSIRION under the trade name LD20. The fluid flow sensor 10 has a side port 110 for receiving liquid sterilizer. In an alternative embodiment without any side port, liquid sterilizer enters the fluid flow sensor 10 through the incoming connector 102. For measuring a gas flow rate, a preferred embodiment of the mass flowmeter 10 is sold by SENSIRION under the trade name SFM3300. A preferred embodiment of the digital computer 5 is sold by the RASPBERRY PI FOUNDATION under the trademark RASPBERRY PI®. An alternate preferred embodiment of the digital computer is sold by ARDUINO SA under the trademark ARDUINO®.

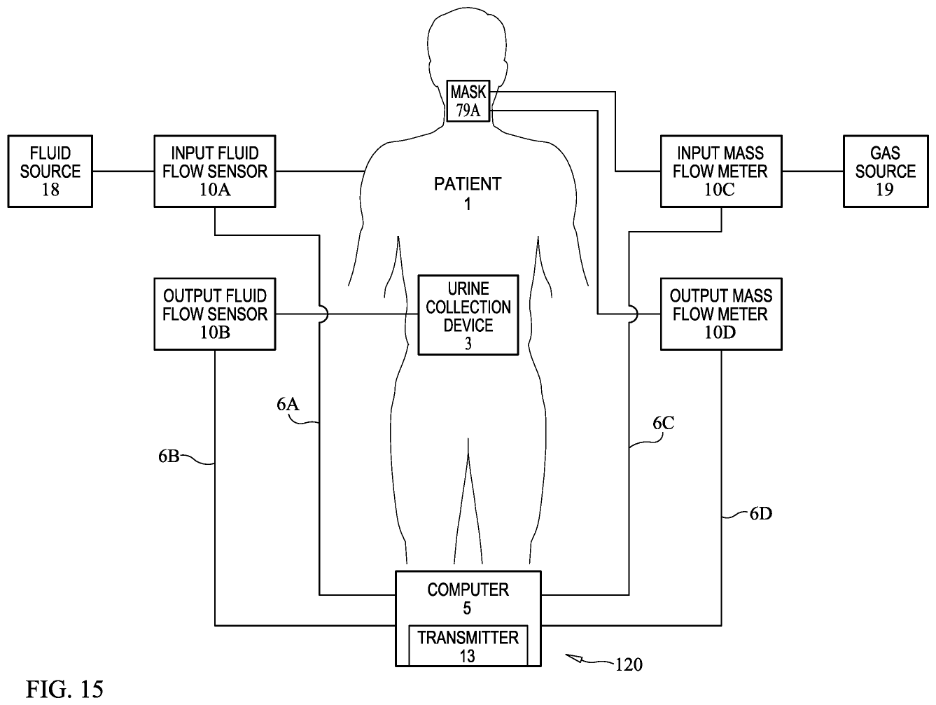

FIG. 15 shows a preferred embodiment of a wireless flowmeter device 120 for measuring fluid input flow rate, urine output flow rate, gas input flow rate, and gas output flow rate. The device 120 includes an input fluid flow sensor 10A, an output fluid flow sensor 10B, an input mass flowmeter 10C, and an output mass flowmeter 10D. The input fluid flow sensor 10A interconnects a fluid source 18 and a patient 1 and measures the flow rate as a function of time of fluid from the fluid source 18 to the patient 1 and generates a signal describing the flow rate as a function of time. A lead 6A carries the signal from the input fluid flow sensor 10A to a digital computer 5. The output fluid flow sensor 10B connects a urine collection device 3, which collects urine from the patient 1, and measures the flow rate as a function of time of urine from the patient 1 and generates a signal describing the flow rate as a function of time. A lead 6B carries the signal from the output fluid flow sensor 10B to the digital computer 5. The input mass flowmeter 10C interconnects a gas source 19 and a mask 79A and measures the flow rate as a function of time of gas from the gas source 19 and generates a signal describing the flow rate as a function of time. A lead 6C carries the signal from the input mass flowmeter 10C to the digital computer 5. The mask 79A further connects to the output mass flowmeter 10D and measures the flow rate as a function of time of gas from the patient 1 and generates a signal describing the flow rate as a function of time. A lead 6D carries the signal from the output mass flowmeter 10D to the digital computer 5. The digital computer 5 includes a wireless transmitter 13 for sending data from the digital computer 5. The digital computer 5 records the flow rate data from the fluid flow sensors 10A and 10B and mass flowmeters 10C and 10D along with time data. Preferably, the time data is a set of times listed in Coordinated Universal Time (UTC) that describes the moments when the flow rate is being recorded from each of the flow fluid flow sensors 10A and 10B and mass flowmeters 10C and 10D. The wireless flowmeter device 120 shown in FIG. 15 provides a closed loop analysis of fluids and gasses.

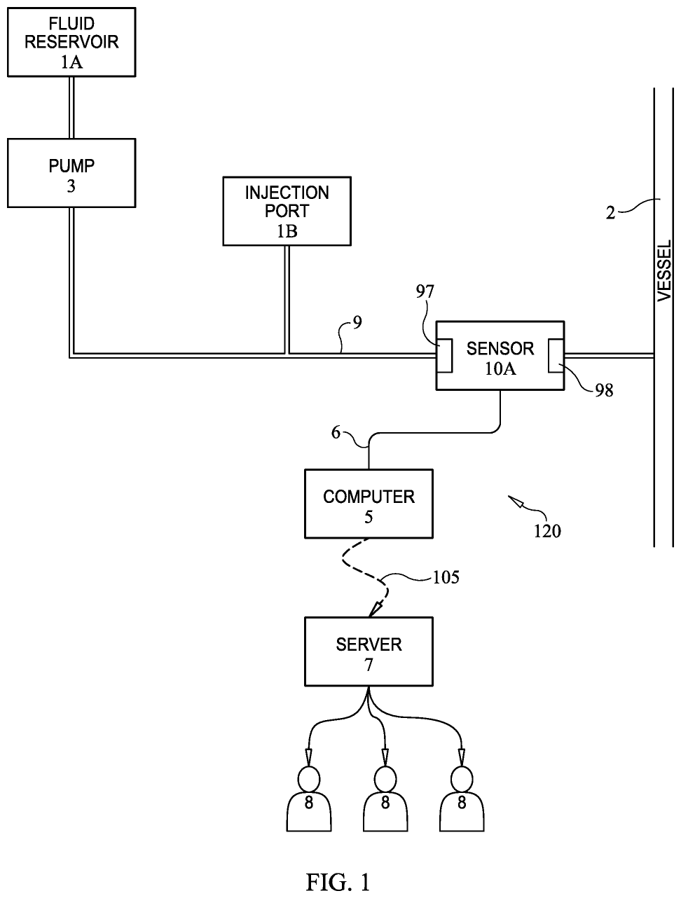

FIG. 1 shows a preferred embodiment of a system for measuring and monitoring the introduction of intravenous fluids into a patient. In this embodiment, a wireless flowmeter device 120 is inserted into a fluid line 9. The wireless flowmeter device 120 includes an input fluid flow sensor 10A that is inserted into the fluid line 9. Intravenous fluids are fed into the fluid line 9 from a fluid reservoir 1A and/or an injection port 1B. The fluid reservoir 1A and the injection port 1B are located upstream of the input fluid flow sensor 10A. A pump 3 harvests fluid from the fluid reservoir 1A and pushes the fluid into a vein 2 of the patient via the fluid line 9. It is possible to inject medication manually into the vein 2 via the injection port 1B and the fluid line 9. The pump 3 has flow control, a back-pressure alarm, a stop flow alarm, and an empty bag alarm, which are not illustrated in the drawing. These alarms are audible alarms. The input fluid flow sensor 10A has an input port 97 and output port 98 for connecting to the fluid line 9 downstream of the pump 3 and the injection port 1B. The input fluid flow sensor 10A is connected to a digital computer 5 by a lead 6. Data from the input fluid flow sensor 10A is encrypted by the digital computer 5 and sent via a wireless network 105 to the server 7 where the data is stored, analyzed, and again sent in the encrypted fashion to end users 8. The input fluid flow sensor 10A measures the flow rate of fluids passing through the input fluid flow sensor 10A, marks a start time of fluid administration, marks an end time of injections, and integrates the flow over time of administration to generate total fluid in. A computer programmed to analyze the flow rate of fluids being input into the patient is connected to the server 7, and detects when the input of fluids stop by analyzing the fluid-flow-rate data received from the input fluid flow sensor 10A, and transmits an alarm to users 8 via a network. A preferred embodiment of an end user is a healthcare provider. The user 8 receives the alarm via a network. So, the user 8 does not need to be present with the patient to receive the alarm.

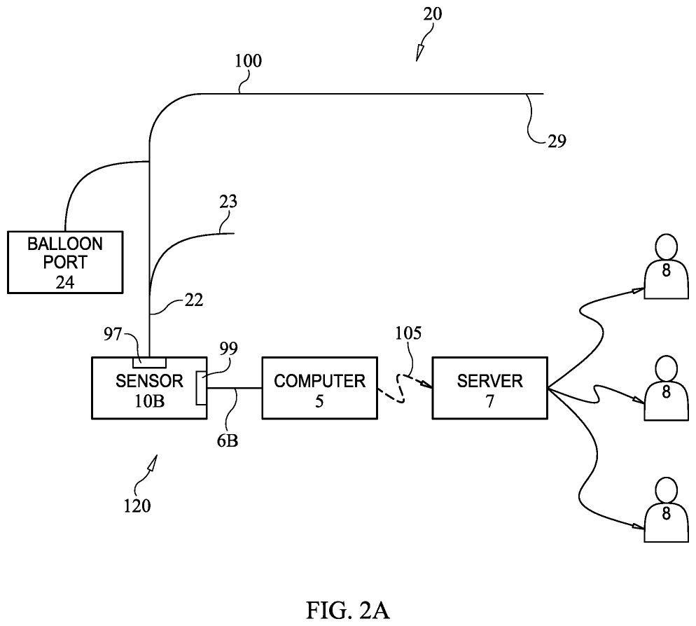

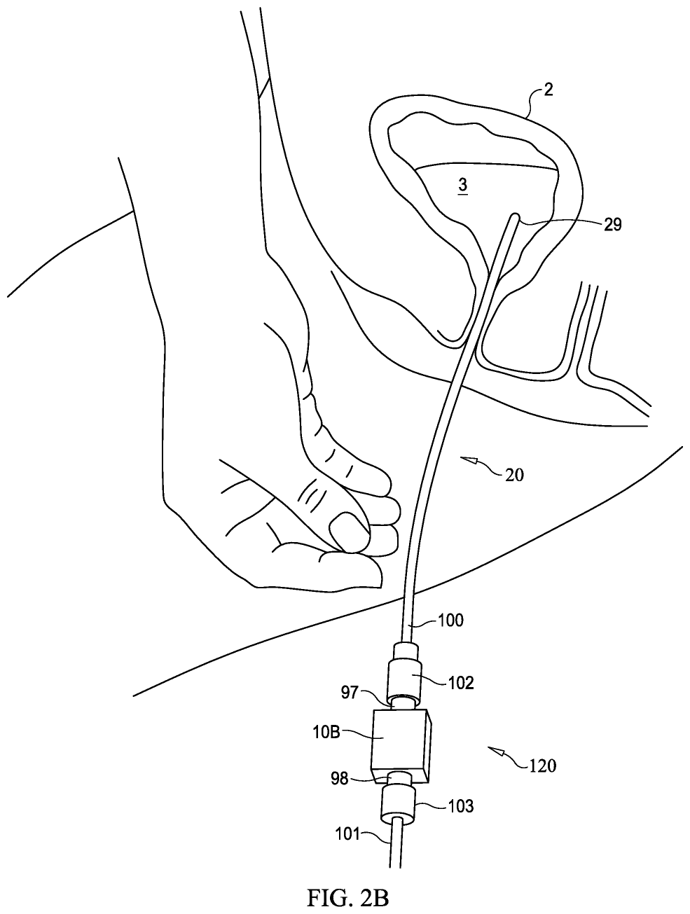

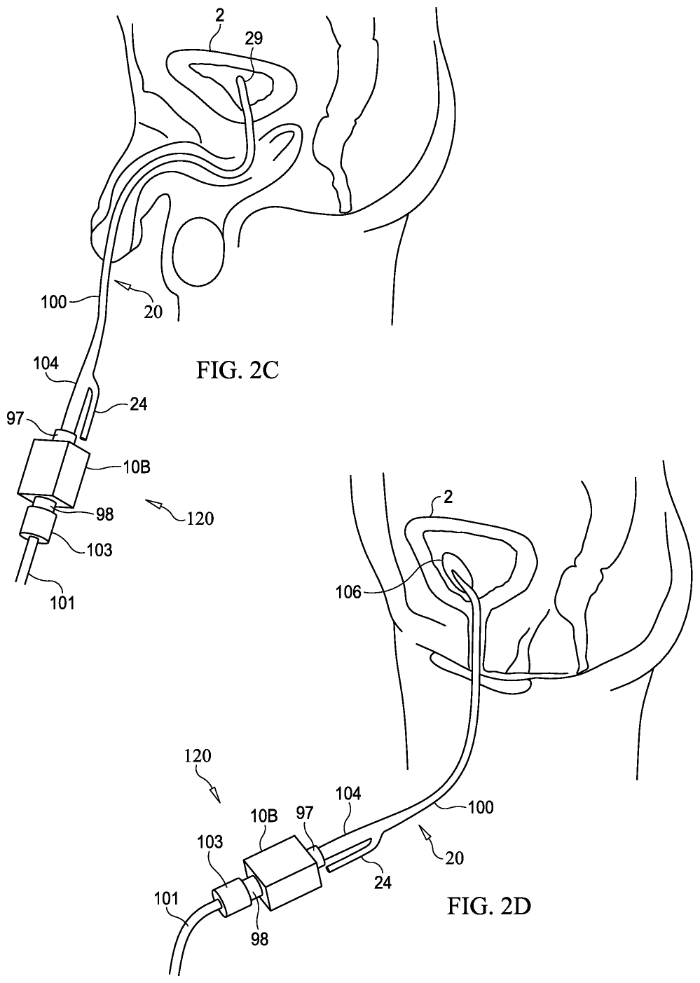

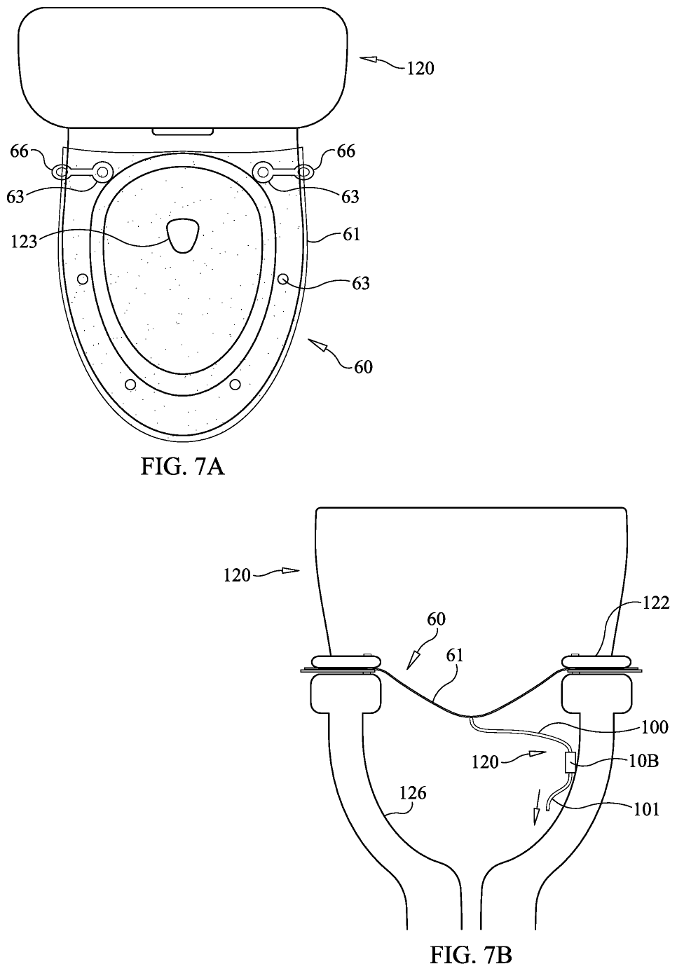

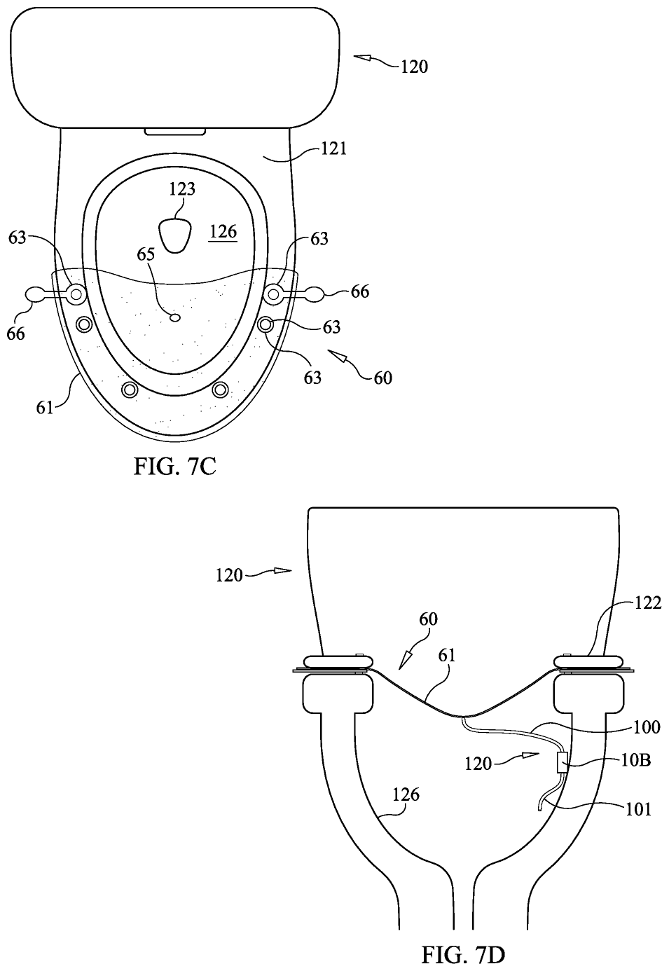

As shown in FIGS. 2A-2D, 3A-3D, 4, 5A-5B, 6D-6E, 7B, 7D, A-7D, and 8A-8B, the wireless flowmeter device 120 can be attached to a variety of urine collection devices 3 (see FIG. 15). As shown in FIGS. 2A-2D, the output fluid flow sensor 10B connects to an indwelling catheter, both male (FIG. 2C) and female (FIGS. 2A, 2B, and 2D). The output fluid flow sensor 10B records the flow rate of urine continuously as urine is collected from the bladder 2.

Additional embodiments of the wireless flowmeter device 120, which are not illustrated in the drawing, include an output fluid flow sensor connected to other drains. Examples of other drains include a JP drain, a thoracentesis, a paracentesis, cholecystostomy, wound vac as examples, not shown here), the automated real-time output if these bodily fluids can be recorded remotely.

FIGS. 2A-2D show a preferred embodiment of a wireless flowmeter device 120 for analyzing urine flow. The wireless flowmeter device 120 includes an output fluid flow sensor 10B. The output fluid flow sensor 10B has an incoming port 97. A connector 22 of the indwelling catheter 20 connects to the incoming port 97. The indwelling catheter 20 has a cannula 100, sterilization port 23, a balloon port 24, and a bladder insertion point 29. The sterilization port 23 is used for chemical sterilization. The sterilization port 23 allows this portion of the indwelling catheter 20 to be reusable in another patient or sterilized when exchanging between catheters in the same patient. The input port 97 is connected to an indwelling catheter 20, the output fluid flow sensor 10B generates a signal that is a function of urine flow rate through the output fluid flow sensor 10B to an output 99. A lead 6B connects the output 99 to a digital computer 5. The digital computer 5 calculates and records urine production as a function of time from the signal from the output fluid flow sensor 10B. A wireless network 105 (and possibly additional network segments) interconnect the digital computer 5 and a server 7. The server 7 stores the data from the digital computer 5. A computer connected to the server 7 calculates a volume of urine below a threshold over a specific time interval from the data stored in the server 7. The computer generates an alarm signal indicating urine retention to the users 8. As shown in FIG. 2B, the output fluid flow sensor 10B has an output port 98. An output connector 103 of an output cannula 101 connects to the output port 98. FIGS. 2B-2D show the bladder insertion point 29 of the indwelling catheter 20 inserted into a bladder 2 of the patient. Urine 3 held in the bladder 2 drains to the output fluid flow sensor 10B via the indwelling catheter 20. FIGS. 2C-2D show a variation of indwelling catheter 20 with a drainage port 104 and balloon port 24. The balloon port 24 is used to inflate balloon 106.

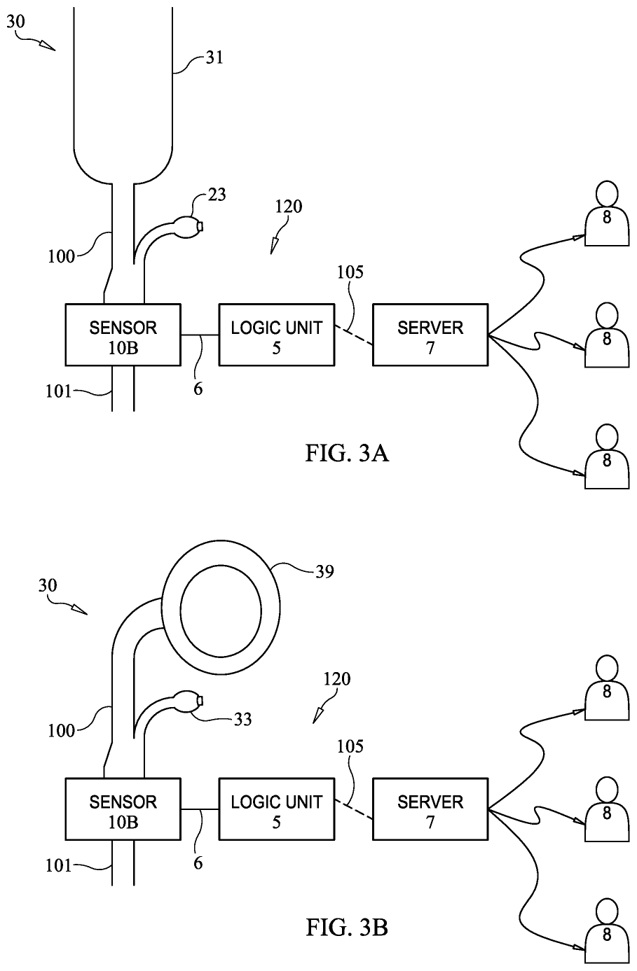

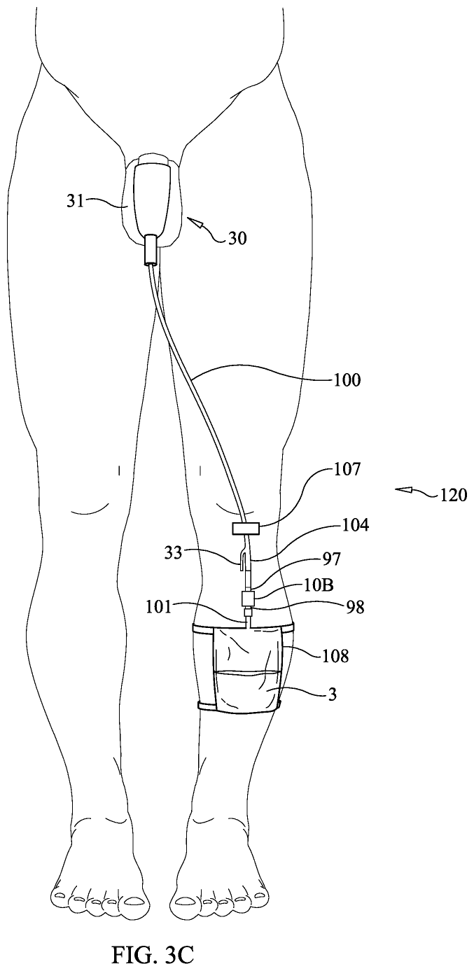

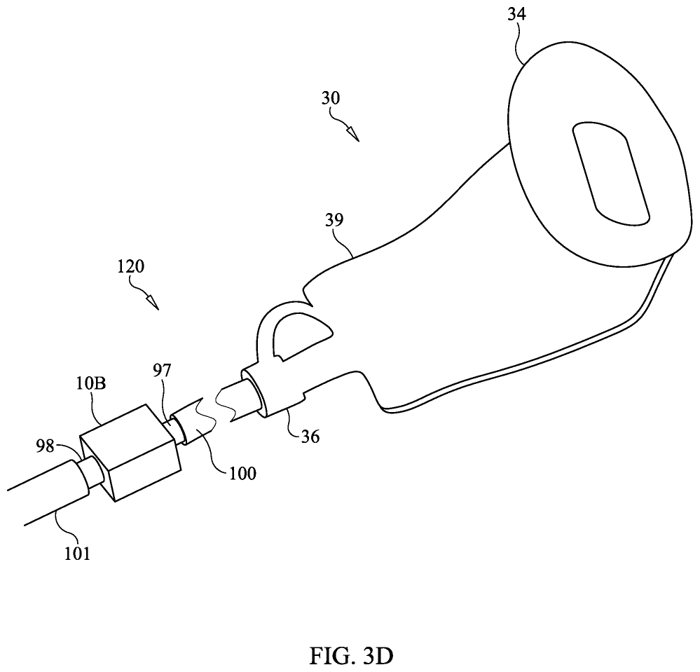

FIGS. 3A-3D show an embodiment of the invention including an external catheter device 30. The external catheter device 30 includes an output fluid flow sensor 10B. A lead 6 interconnects an output fluid flow sensor 10B and a digital computer 5. The digital computer 5 connects to a server 7 by a wireless connection 105. The server 7 send alarms and messages to users 8. FIGS. 3A and 3C show an embodiment with a condom 31. Urine drains from the condom 31 via an input cannula 100 to the output fluid flow sensor 10B. A sterilization port 23 is connected to the input cannula 100. The output fluid flow sensor 10B has an output port 98, An output cannula 101 interconnects the output port 98 and a urine collection pouch 108. Adhesive tape 107 attaches the input cannula 100 to the patient's leg. The embodiment shown in FIGS. 3A and 3C works with male anatomy and includes a male condom 31. The embodiment shown in FIGS. 3B and 3D works with female anatomy and includes a urinary pouch 39. The urinary pouch 39 includes an adhesive gasket 34. The urinary pouch 39 has an outlet 36 connected to the input cannula 101.

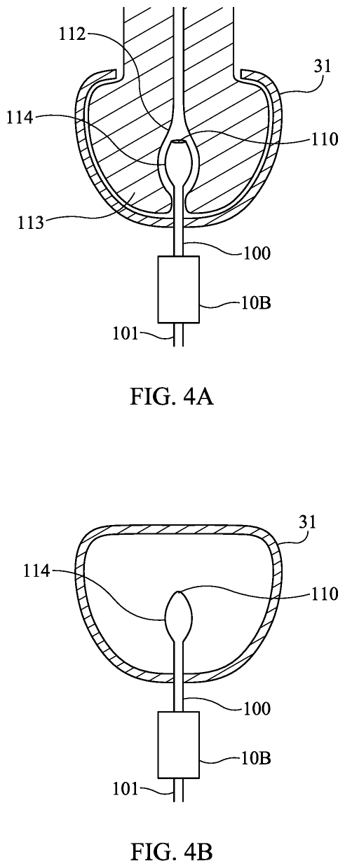

FIGS. 4A and 4B shows an embodiment with an indwelling catheter device inserted in a fossa navicularis 116. The indwelling catheter device has an input cannula 100 ending with a bulb 114 inserted in the fossa navicularis 116 of the urethra 112, which allows for good physical attachment, non-dependent on the size of the penis, overcoming common problem of detachment. The bulb 114 is preferably one to two centimeters long. The bulb is preferably made of plastic material such as silicone. In addition, a short and relatively loosely attached condom 31 overlies the glans 113. A distal tip 110 is open when inserted in the fossa navicularis 116, as shown in FIG. 4A. The opening preferably spans two to three millimeters. As shown in FIG. 4B, the distal tip 110 is closed before being inserted. The input cannula 100 is connected to an output fluid flow sensor 10B. The output fluid flow sensor 10B measures a flow rate of urine through the fluid flow sensor 10B.

In an embodiment that is not shown, very additional thin tubing can be inserted directly into the patient's bladder making it like indwelling Foley catheter but avoiding traumatic insertion and injury. This is particularly important in patient with prostate strictures and penile implants and otherwise pathologically narrowed urinary tract.

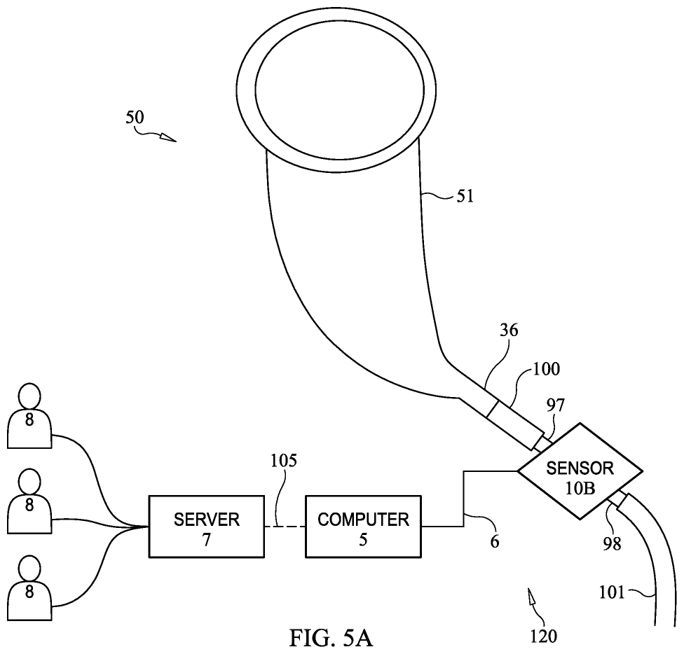

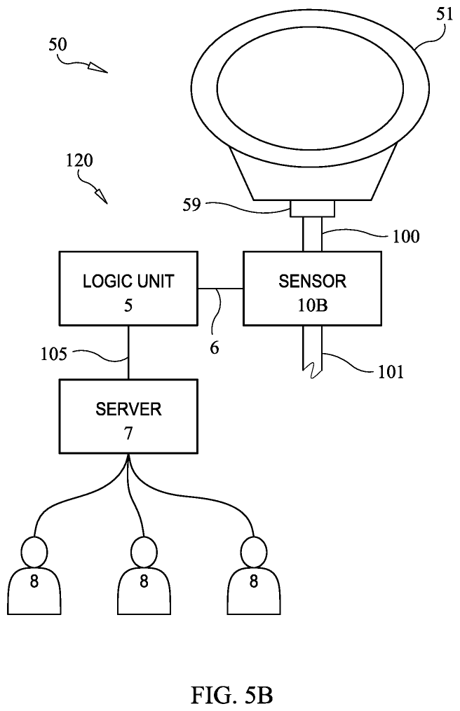

FIGS. 5A-5B show a system including a collection hat device 50 for measuring a flow rate of urine from a patient. The system includes a wireless flowmeter device 120. The collection hat device 50 includes a collection hat 51. A collection hat device 50 has an outlet 36 connected to an input cannula 100. The input cannula 100 is connected to an input port 97 of an output fluid flow sensor 10B. A patient urinates into the collection at device 50, which drains into the output fluid flow sensor 10B, which measure the flow rate of the urine. The output fluid flow sensor 10B is connected to a digital computer 5 with a lead 6. The digital computer 5 records the data from the fluid flow sensor 10B and transmits it over a network that includes a wireless segment 105 to a server 7. A computer connected to the server 7 reads the data and alarms users 8 when the urine flow rate falls below a given minimum. Sterilization can be done by pouring sterilization fluid directly into collecting hat 51.

Alternative embodiments of urine collection devices, which are not shown in the drawing, include urinals, rigid collection hats, and bedside commodes.

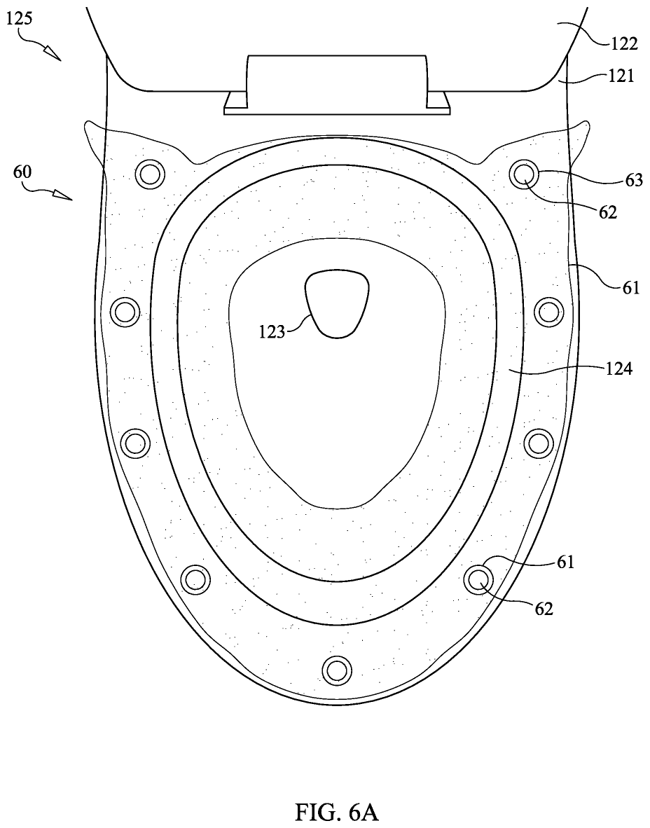

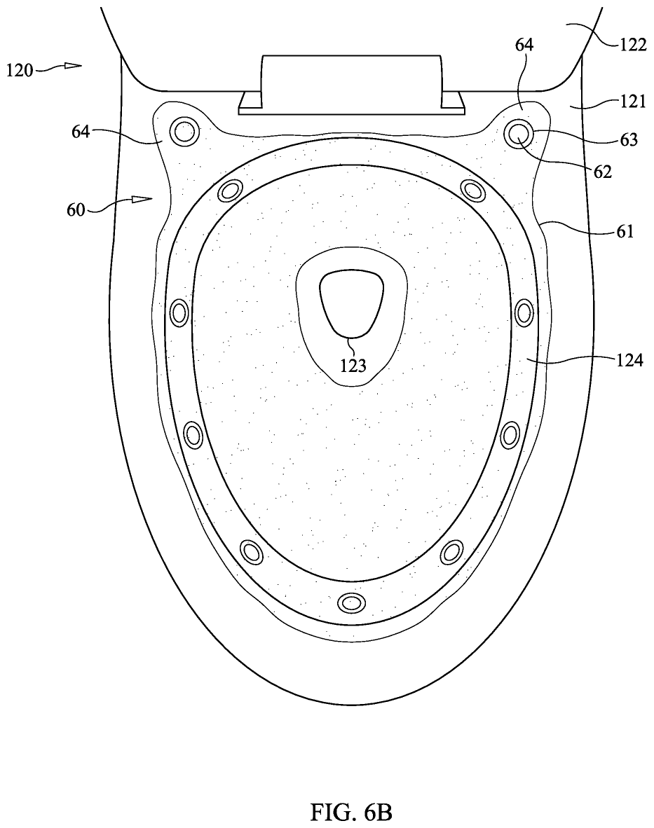

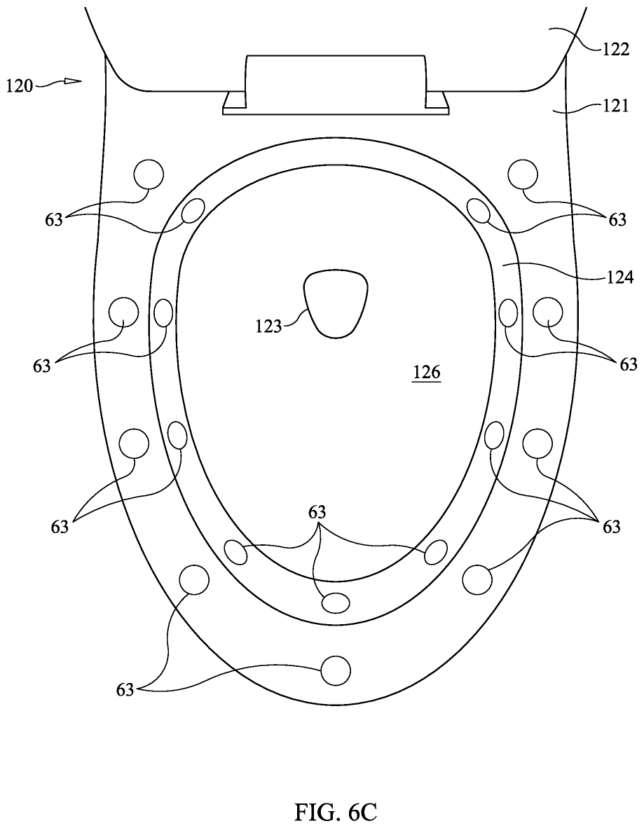

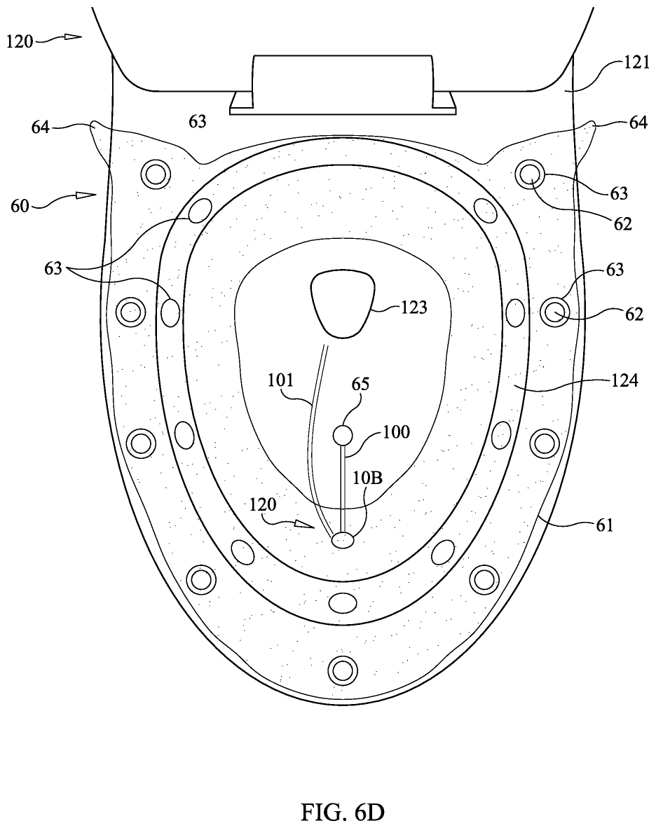

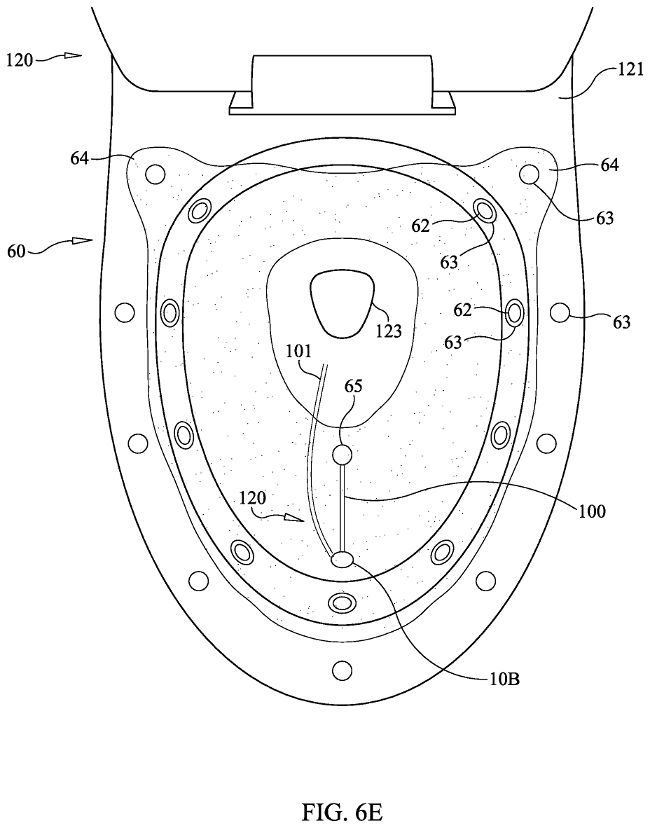

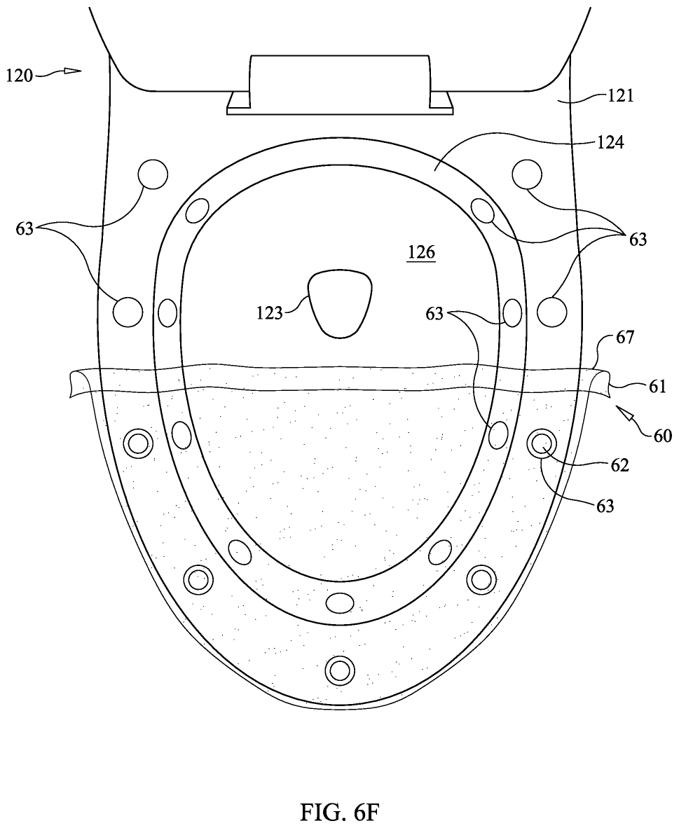

FIGS. 6A-6F show a preferred embodiment of a urine collection unit 60 that includes a sheet 61 made of flexible and thin plastic that is to be used with a toilet 125. The urine is collected into container that can be made of plastic with adequate physical properties (pliable, biocompatible material): for example, polypropylene, latex, silicone or other. Several possible models of these devices presented in FIGS. 6A-6F.

In an embodiment shown in FIGS. 6A-6B, 6D the urine collection device 60 is composed of two parts. The top unit serves as an attachment to the bowl and can be permanently attached to the toilet top rim 121 (FIG. 6A) or internal side rim 124 (FIG. 6B) of the ceramic bowl with adhesive. The top unit contains magnetic disks 63 that can be affixed with adhesives or be placed in pockets. The magnetic disks 63 are attached permanently. The lower, disposable collecting unit contains iron disks 62 that can be attached to the magnetic disks 63 of upper part. Six magnetic disks 63 and four iron disks 62 is the minimal number that is needed for proper function of the collection unit. The device allows adjustment of the collection area of the bowl to be full position as shown in FIG. 6D, which allows urine collection while standing, or part position as shown in FIG. 6F, which allows urination while sitting and separation of feces from urine by appropriate positioning of the iron disks 62. As shown in FIGS. 6D-6E, plastic ears 64 help to achieve better grip when changing position of the lower part of the unit. Alternatively, the urine collection container 60 can be a one-unit device directly attached to the ceramic bowl with magnet/magnet or iron/magnet interaction.

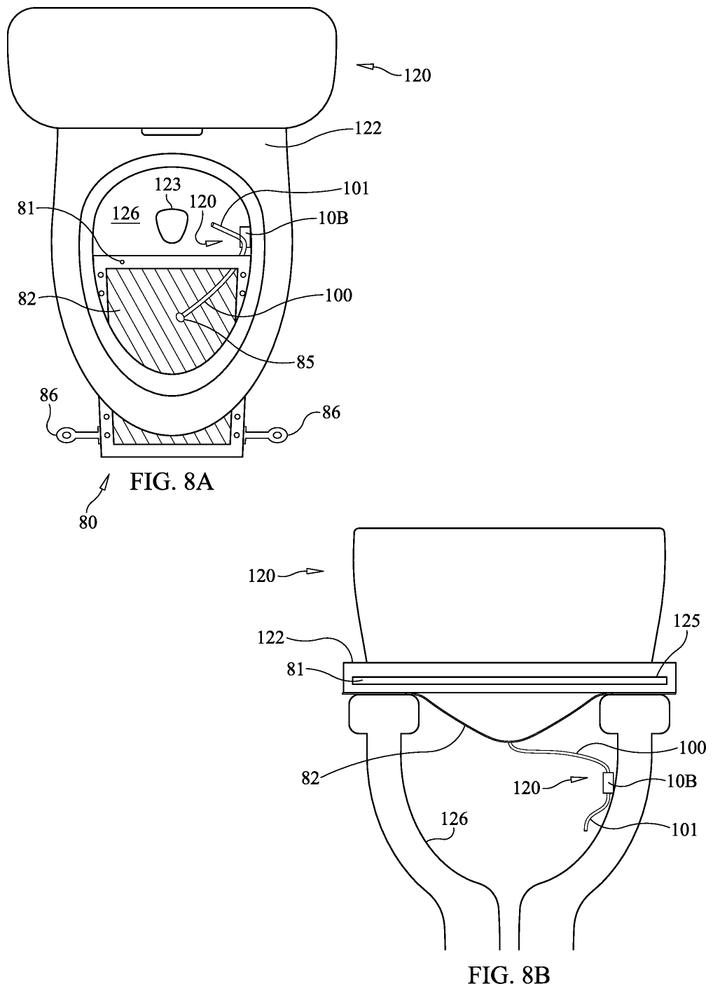

FIGS. 8A-8B show an embodiment of a urine collection device having specially configured toiled seat 122 with integrated moving tray 80. The integrated moving tray 80 can slide horizontally, forward and backward, in a slot 125. A urine collection bag made of metal or plastic with magnets/magnets, magnets/iron, or adhesive is attached to the integrated moving tray 80. An outlet 85 of the urine collection bag 82 is connected to an input cannula 100 that carries urine to the output fluid flow sensor 10B and further into collection bowl. The ears 86 help to move the integrated moving tray 80 forward and backward. FIG. 8A shows the integrated moving tray 80 in half inserted position.

In the embodiments shown in FIGS. 6-8, the sheet 61 is preferably reusable or disposable. As shown in FIGS. 6D, 6E, 7B, 7D, 8A, and 8B, the output flow sensor 10B is attached to the toilet bowl 126, above water level, and to the input cannula 100, which is connected to the outlet 85 of the urine collection unit 60. The urine, after passing through the output fluid flow sensor 10B, is led by the output cannula 101 to the toilet bowl 126.

Again referring to FIG. 9 shows a preferred embodiment of a wireless flowmeter device 120 that is used as a wireless transcutaneous capnography probe. In this embodiment, a mass flowmeter 10 is made to function in wireless portable fashion. As shown in FIG. 15, the mass flowmeters 10C and 10D are connected to a digital computer 5 with detachable leads 6C and 6D, respectively.

The data from the mass flowmeters 10C and 10D are encrypted and sent by the digital computer 5 to a server 7 where it is stored, analyzed, and again sent in the encrypted fashion to end users 6. The wireless flowmeter device 120, which is embodied as a wireless transcutaneous capnography probe, measures real-time carbon-dioxide levels, oxygen levels, and heart rate. If the values fall outside of desired range, a computer connected to the server sends a warning signal to end users 8, remotely, if the computer calculates that the value(s) are outside a specified range.

FIG. 10 shows a preferred embodiment of a wireless oxygen flowmeter 70. An oxygen delivery device (ODD) 79, for example a patient mask or patient nasal cannula, delivers oxygen to the patient 1. The input mass flowmeter 10C is inserted into a gas flow line 72 connected to an oxygen source 71. A lead 6C connects the input mass flowmeter 10C to the digital computer 5 and carries the flow rate data from the input mass flowmeter 10C to the digital computer 5. The digital computer 5 encrypts the data from the input mass flowmeter 10C and sends the encrypted data to a server 7 at least in part over a wireless connection 105 and the Internet 109, which is connected by a gateway to the wireless connection 105. The server 7 stores the data for recall and analysis by users 8 and an attached computer 114. The server 7 encrypts the data stored therein while the data is at rest. The computer 114 is programmed to calculate real time oxygen usage by integrating the flow rate over the time observed. If the computer 114 calculates that the oxygen volume and/or oxygen flow rate fall outside a desired range, the computer sends an alarm over a network to the users (e.g. a healthcare provider) 8, who may be remote from the patient 1.

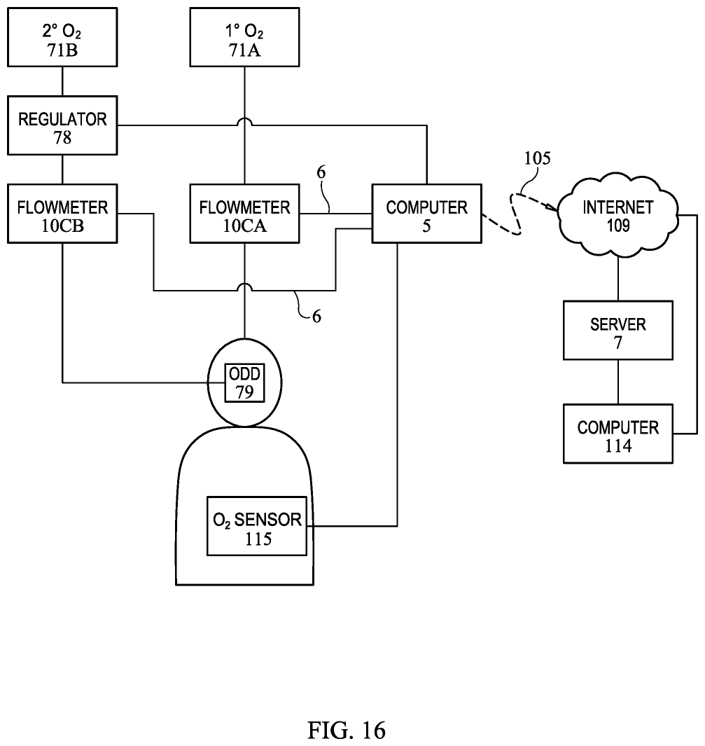

FIG. 16 shows a preferred embodiment of a system for supplying oxygen based on analysis of a closed loop. In the embodiment shown, a concentrator is a primary oxygen source 71A and an oxygen tank is a secondary (backup) oxygen source 71B. A regulator 78 controls the flow rate of oxygen from the secondary oxygen source 71B. Each oxygen source 71A and 71B have a respective mass flowmeter 10CA and 10CB. Each mass flowmeter 10CA and 10CB transmits its own signal based on the oxygen flow rate measured by the particular mass flowmeter 10CA or 10CB to the digital computer 5. In addition, a transcutaneous blood oxygen sensor 115 sends a signal to the digital computer 5 that is a function of the blood oxygen level of the patient 1. The digital computer 5 transmits a signal describing the data from each of the mass flowmeters 10CA and 10CB across a wireless network segment 105 via the Internet 109 to a server 7, where the data is stored. A computer 114 reads the data from the server 7 and sends a signal to the digital computer 5 to increase or decrease the oxygen flow through the regulator 78 in order to adjust the blood oxygen level of the patient 1 to a set point or set range. The computer 114 further analyzes the data of the patient 1 to generate oxygen usage patterns.

In an embodiment like that shown in FIG. 16, instead of having a secondary oxygen source 71B, a flow rate of a gas source other than oxygen, for example, nitrogen, carbon dioxide, carbon monoxide, helium, etc. can be measured.

Wireless Flowmeter Device Equipped Coupled with Spectrophotometer, PH Meter, and Conductometer

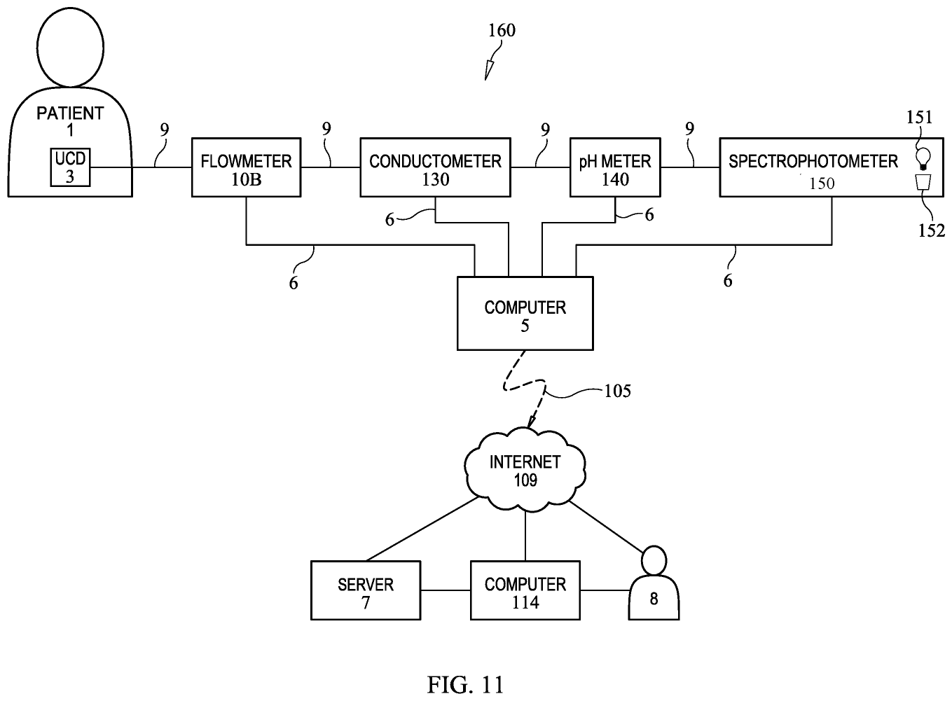

FIG. 11 shows a wireless multi analytical device 160 for analyzing urine and other body fluids, used in medicine. A urine collection device (UCD) 3 collects urine from the patient 1. A wireless output flowmeter 10B receives urine from the UCD 3 and measures a flow rate of the urine through the wireless output flowmeter 10B. A conductometer 130 receives urine from the wireless output flowmeter 10B and measures a conductance of the urine. The conductometer 130 has an output and generates a signal that is a function of the conductance. The conductometer 130 passes the signal to the output of the conductometer 130. A pH meter 140 receives urine from the conductometer 130 and measures a pH of the urine. The pH meter 140 has an output and generates a signal that is a function of the pH being measured. The pH meter 140 passes the signal to the output of the pH meter. A spectrophotometer 150 receives urine from the pH meter 140. The spectrophotometer 150 measures absorbance and/or fluorescence of the urine. The spectrophotometer 150 generates a signal that is a function of the absorbance. The spectrophotometer 150 has an output. The spectrophotometer 150 passes its signal to the output. A fluid line 9 interconnects the UCD 3 and the OFFS 10B, the OFFS 10B and the conductometer 130, the conductometer 130 and the pH meter 140, and the pH meter 140 and the spectrophotometer 150. The body fluid (e.g. urine or any other) runs through the fluid line 9. The spectrophotometer conduit 9A is made of an inert material that does not absorb light in the target wavelength; preferred materials include glass, polyvinyl, and silicone. The spectrophotometer 159 measures light intensity over wavelength (spectrophotometry, fluorometry). The pH probe 130 and the conductance probe 140 are in direct contact with the body fluid and are inserted in line. The spectrophotometer 150 is an outside sensor consisting of two parts: a light source 151 and a photosensor 152. The light source 151 emits light (broad spectrum for spectroscopy, or defined excitation length for fluoroscopy) that passes through the body fluid and the photosensor 152 generates a signal that is a function of the intensity of the light after passing through the body fluid. In a preferred embodiment, the light source 151 and the photosensor 152 are clipped on opposing sides of the fluid line 9. Preferred embodiment of the photosensor 152 are sold under the trademarks NSP32M by NANOA and MINI-SPECTROMETER MICRO SERIES by HAMAMATSU®. The light source 151 is preferably a broad spectrum emitter, for example tungsten or xenon, or laser. In an alternate embodiment in which the light source 151 emits white light, the photosensor 152 measures absorbance across the entire spectrum. Each of the outputs of the OFFS 10B, Conductometer 130, pH meter 140, and spectrophotometer 150 has respective leads 6 that connects it to the digital computer 5. The digital computer 5 relates the signals to each other at given times. The digital computer 5 provides power to OFFS 10B, Conductometer 130, pH meter 140, and spectrophotometer 150 via each respective lead 6. The digital computer 5 encrypts and transmits the encrypted signals at least in part via a wireless connection 105 to a server 7. The OFFS 10B, Conductometer 130, pH meter 140, and spectrophotometer 150 can have any ordered in series along the fluid line 9. In a preferred embodiment, flow through the fluid line 9 is bidirectional. A computer 114 is connected to the server 7. The computer generates an alarm based on data from at least one of the sensors (i.e. the OFFS 10B, the conductometer 130, the pH meter 140, and the spectrophotometer 150). In an alternative embodiment, the computer 114 generates an alarm based on data from more than one of the sensors. The computer 114 transmits the alarm to a user 8.

A preferred method of using the wireless multi analytical device 160 shown in FIG. 11 is to measure glomerular filtration rate (GFR). A first step of the method involves injecting a renally secreted, not absorbed, inert dye into a patient's bloodstream. As the patient's kidneys work, the dye will be secreted into urine of the patient 1. The output fluid flowmeter 10B measures the flow rate of urine from the patient 1. The light source 151 of the spectrophotometer 150 is set to a wavelength absorbed by the dye. The photosensor 152 measures the absorbance of the urine. From the flow rate data and the absorbance, the computer 114 calculates the glomerular filtration rate of the patient 1. The computer 114 generates an alarm when the glomerular filtration rate falls below a threshold. The computer 114 transmits the alarm to the user 8.

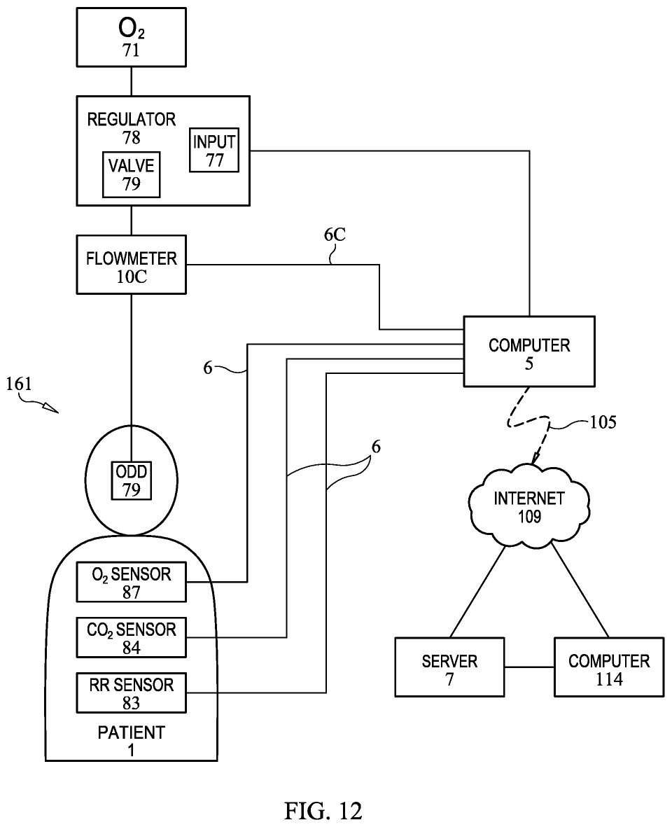

FIG. 12 shows a preferred embodiment of an open loop respiratory device 71 for monitoring respiration of a patient 1. An oxygen source (O₂) 71 supplies oxygen for a patient 1. A regulator 78 for adjusting a flow rate of the oxygen connects to the oxygen source 71. The regulator 78 includes a valve 79 that opens or closes to increase or decrease the flow rate of oxygen through the regulator 78. The regulator 78 has an input for receiving signals. When the regulator receives an open signal, the regulator 78 opens the valve 79 An input mass flowmeter 10C for measuring a flow rate of the oxygen connects to the regulator 78. An oxygen delivery device (ODD) 79 connects to the input mass flowmeter 10C. Two preferred embodiments of the oxygen delivery device are a mask and a nasal cannula. The input mass flowmeter 10C transmits a signal that is a function of the flow rate to the digital computer 5 via the lead 6C. An oxygen saturation sensor 87 for measuring a percentage of oxygen saturated hemoglobin in blood of the patient 1 is disposed on the patient 1. The oxygen saturation sensor 87 generates a signal that is a function of the percentage of oxygen saturated hemoglobin and transmits the signal to the digital computer 5 via a lead 6. A carbon dioxide sensor 84 for measuring a concentration of carbon dioxide in the blood of the patient is disposed on the patient 1. The carbon dioxide sensor 84 generates a signal that is a function of the carbon dioxide concentration and transmits the signal to the digital computer 5 via a lead 6. A respiratory rate sensor 83 for measuring frequency of breathing of the patient 1 is disposed on the patient 1. The respiratory rate sensor 83 generates a signal that is a function of the respiratory rate and transmits the signal to the digital computer 5 via a lead 6. In additional embodiments, which are not shown, then open loop respiratory device includes additional sensor for measuring respiratory-related properties of the patient including a heart rate sensor, a blood pressure sensor, a central venous pressure sensor, and a temperature probe, all of which transmit signals describing the measured property to the digital computer 5. The digital computer 5 uses a wireless connection 105 and the Internet 109 to transmit data to a server 7. The server 7 stores the data describing the respiratory data along with time data that is used to relate the different signals to each other. A computer 114 analyzes the data from at least one of the input mass flowmeter 10C, the oxygen saturation sensor 87, the carbon dioxide sensor 84 and the respiratory rate sensor 83. When the computer detects that the data outside of a set range, the computer 114 generates a signal to change the oxygen flow rate to the patient 1. The computer 114 transmits the signal to the digital computer 5, which relays the signal to the input 77 of the regulator 78. The regulator then increases or decreases the flow rates based on the type of signal received by the input 77. The valve 79 does not necessarily completely open and completely close based on the signal received by the input 77; rather the valve may only partially open or partially close to increase or reduce the oxygen flow reaching the patient 1.

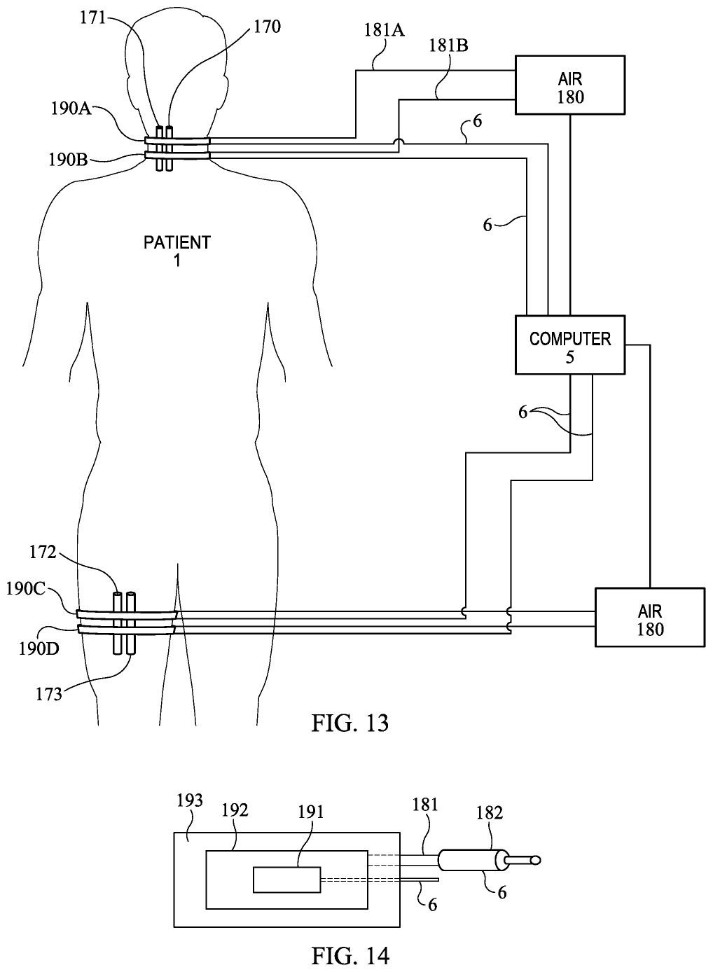

FIGS. 13 and 14 show a wireless noninvasive combined central venous pressure meter and arterial pressure meter 162. The wireless noninvasive combined central venous pressure and arterial pressure meter 162 measures accurately, in real time, central venous pressure and arterial pressure. The meter 162 includes two pairs of central blood vessel sensors 190: the right superior vena cava sensor 190A, the right carotid artery sensor 190B, the right femoral vein sensor 190C, and the right femoral artery sensor 190D. As shown in FIG. 14, each central blood vessel sensor 190 includes an ultrasound probe 191. The ultrasound probe 191 measures changes of geometry of a central artery (e.g. right carotid artery 170 and right femoral artery 172) and a central vein (e.g. right superior vena cava 171 and right femoral vein 173) caused by applying external pressure. A bladder 192 fills with air to apply pressure against the underlying blood vessel. The ultrasound probe 191 generates a signal that is a function of the change in geometry. The ultrasound probe 191 transmits its signal to a digital computer 5 via a lead 6. A stretchable strap 193, which is secured with adhesive, holds the ultrasound probe 191 and bladder 192 in place. The ultrasound probe 191 is centered on vessels (170 and 171; or 172 and 173) by an operator. The operator will see anatomy displayed on a monitor, which is not shown. The operator can be assisted remotely by experienced technician if needed. Once the vessels are visualized and identified, the ultrasound probe 191 is fixed into position and ready to use.

The meter 162 measures central pressures as follows. Air from an air pump 180 inflates the bladder 192. The inflation of the bladder 192 presses the ultrasound probe 191 down externally (against the skin) with defined pressures. The air pump 180 the bladder 192 to a preselected value. As shown in FIG. 14, a pressure meter 182 measures the pressure in the bladder 192. The pressure meter transmits a signal that is a function of the measured air pressure to the digital computer via a lead 6. The ultrasound probe 191 measures the deformation (cross section) and wall motion of the vessels during a complete heartbeat. The digital computer calculates the central arterial pressure and venous pressure from the deformation data from the ultrasound probe 191 using prior-art algorithms.

Wireless Oxygen Flow Sensor and Devices Equipped with Wireless Oxygen Flow Sensor.

FIG. 10 shows a preferred embodiment of a wireless, wearable, real time oxygen flowmeter, which is also referred to as an oxygen flow sensor, was created to address and solve significant problems associated with oxygen therapies. The oxygen gas is flowing via gas flow line 72 and runs through input mass flowmeter 10C continuing to the oxygen delivery device (ODD) 79. Therefore, functionally it can be considered “in” sensor. The oxygen flow data is collected from any oxygen or gas delivery line equipped with an input mas flowmeter 10C with lead 6C and transmitted to the digital computer 5. Digital computer 5 is a cell phone size wearable unit capable of collecting and transmitting data further as well as powering the input mas flowmeter 10C. The signal is sent wirelessly (at least in part) to the server 7 where the signal is stored, analyzed, and analyzed by performing additional operations. The input mass flowmeter 10C is capable of measuring flow rate of other compatible gases as nitrogen, carbon dioxide, carbon monoxide, helium etc. The input mas flowmeter 10C is reusable and able to withstand medical sterilization.

The wireless gas “input” flow sensor can be coupled with an output mass flowmeter 10D to create an input/output system, “out”, away from the patient 1 is shown. As shown in FIG. 12, preferred sensors include an oxygen saturation probe 87, dissolved oxygen concentration, a carbon dioxide sensor 84, and a respiratory rate sensor 83. The sensors 83, 84, and 87 measure values and transmits the data to the digital computer 5, which relays the data to the server 7 in similar fashion as the input mass flowmeter 10C. As shown in FIG. 10, when both an input mass flowmeter 10C and an output mass flowmeter 10D in addition to previous functions, then the computer 114 sets alarms based on a dose/response relationship. For example, what concentration of oxygen is needed to achieve certain oxygen saturation or carbon dioxide levels. Another type of relationship would be oxygen saturation to carbon dioxide depending on oxygen flow rate.

Spectrometry of Fluids Including Blood

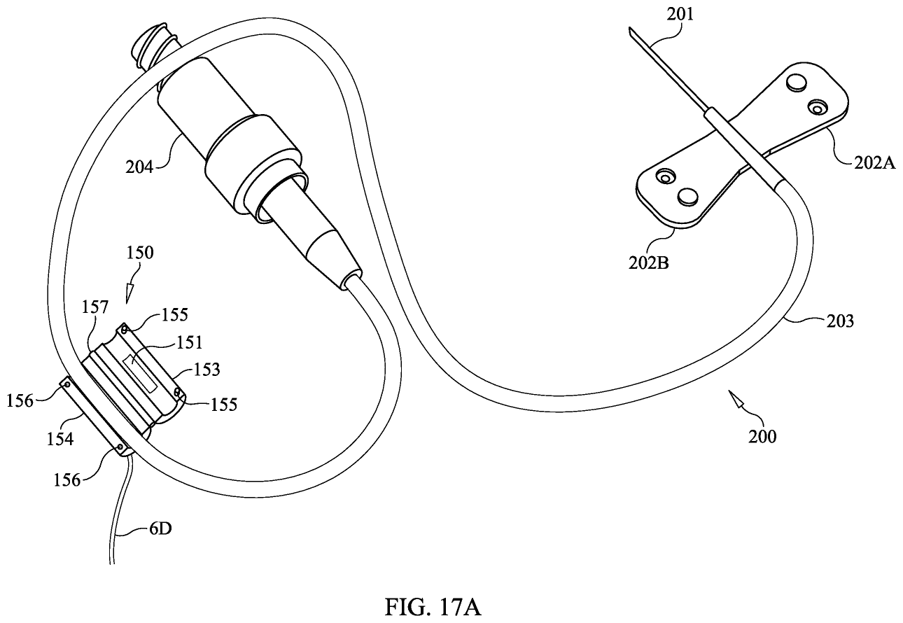

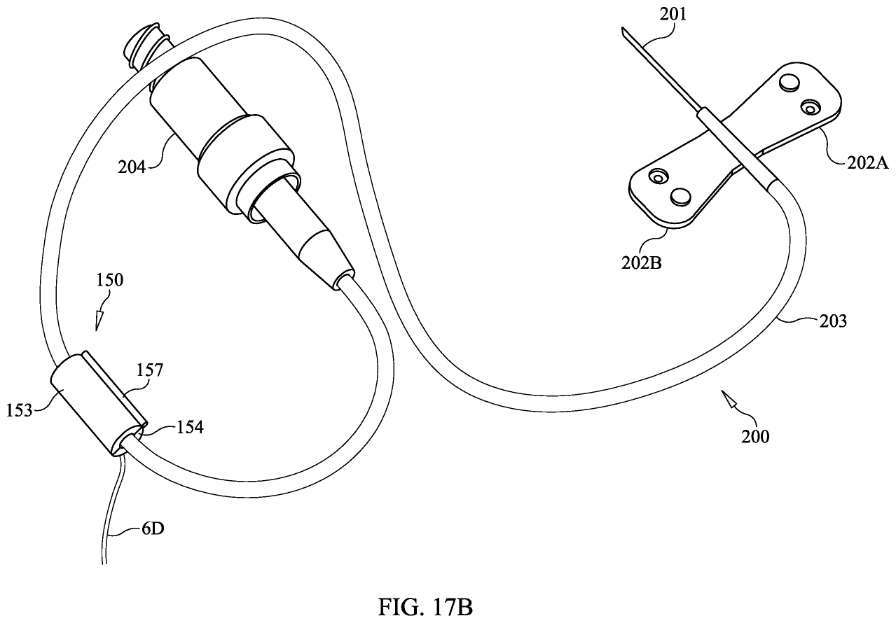

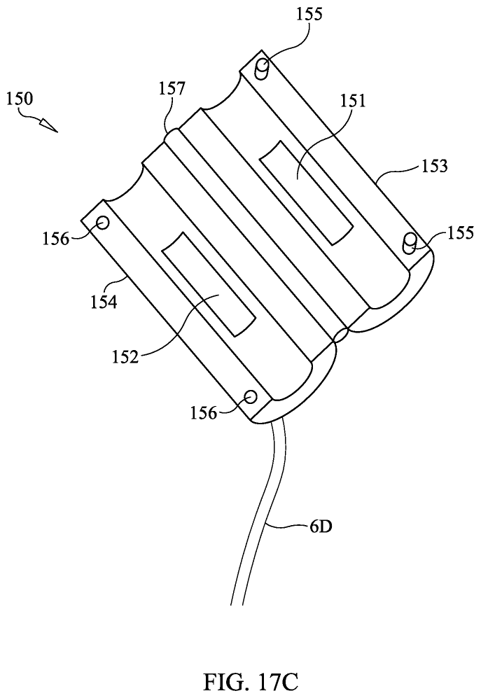

FIGS. 17A-17C show a preferred embodiment of a spectrophotometric phlebotomy device. The spectrophotometric phlebotomy device includes a winged infusion device 200. The winged infusion set 200 has a hypodermic needle 201 that inserts into a vein of the patient. The hypodermic needle 201 has two wings 202A and 202B for securing the needle 201 in the vein. A flexible small-bore transparent tubing 203 is connected to a proximal end of the hypodermic needle 201.

A spectrophotometer 150 has two hemi annular cylinders 153 and 154. The first hemi annular cylinder 153 has a light source 151, such as a LED or laser. The second annular cylinder 154 has a photosensor 152. The light source 151 emits light at a wavelength that is absorbed by hemoglobin. The photosensor 152 measures absorbance by the hemoglobin in the flexible small-bore transparent tubing 203. A lead 6D is used to connect the spectrophotometer 150 to a digital computer 5. A preferred embodiment of a mini-spectrometer is sold under the trademark HAMAMATSU C12666MA. A hinge 157 connects the hemi annular cylinders 153 and 154. Pegs 155 snap into the sockets 156.

What is claimed is:

1. A device for monitoring fluid or gas being input to or output from a patient, comprising:
a fluid flow sensor or mass flowmeter generating a signal, the signal being a function of a flowrate of the fluid or gas through said fluid flow sensor;
a digital computer being connected to and receiving the signal from said fluid flow sensor or mass flowmeter, said digital computer generating a wireless signal from the signal; and
a wireless transmitter being connected to said digital computer, receiving the wireless signal from said digital computer, and transmitting the wireless signal.

2. The device according to claim 1, further comprising:
an output of said fluid flow sensor or mass flowmeter for relaying the signal from said fluid flow sensor or mass flowmeter;
an input of said digital computer for receiving the signal; and
a lead interconnecting said output of said fluid flow sensor or mass flowmeter and said input of said digital computer, said lead relaying the signal from said output of said fluid flow sensor or mass flowmeter to said input of said digital computer.

3. The device according to claim 1, further comprising:
a urine collection device for collecting urine from the patient; and
said fluid flow sensor or mass flowmeter being an output fluid flow sensor generating the signal based on a flowrate of the urine through said output fluid flow sensor; and
an input of said output fluid flow sensor being connected to said urine collection device and receiving the urine from said urine collection device.

4. The device according to claim 1, further comprising:
a fluid source for delivering fluid to a patient;
said fluid flow sensor or mass flowmeter being an input fluid flow sensor generating the signal based on a flowrate of the fluid through said input fluid flow sensor;
an input of said input fluid flow sensor being connected to said fluid source and receiving the fluid from said fluid source;
an output of said input flow sensor for outputting the fluid from said input flow sensor; and
a fluid delivery device for delivering the fluid to the patient.

5. The device according to claim 1, further comprising:
a gas source for delivering a volume of a gas to a patient;
said fluid flow sensor or mass flowmeter being an input mass flowmeter generating the signal based on a flowrate of the gas through said input mass flowmeter;
an input of said input mass flowmeter being connected to said gas source and receiving the gas from said gas source;
an output of said input mass flowmeter for outputting the gas from said input mass flowmeter; and
an oxygen delivery device for delivering the gas to the patient being connected to said output.

6. The device according to claim 1, further comprising:
an exhalation collection device for receiving a gas exhaled by the patient;
said fluid flow sensor or mass flowmeter being an output mass flowmeter generating the signal based on a flowrate of the gas through said output mass flowmeter; and
an input of said output mass flowmeter being connected to said mask and receiving the gas from said exhalation collection device.

7. A system for measuring and monitoring fluid or gas input or output, comprising:
the device according to claim 1;
a server receiving the signal transmitted from said wireless transmitter and storing the signal; and
a computer being connected to said server and reading the signal from said server, said computer deriving data from the signal and generating an alarm when the data exceeds a parameter, and transmitting the alarm to a user.

8. The system according to claim 7, further comprising:
a sensor for measuring health data of the patient and generating a sensor signal, the sensor signal being a function of the health data over time, said sensor being connected to said digital computer;
said wireless transmitter transmitting the sensor signal to said server;
said server storing the health data received from said wireless transmitter; and
said computer generating a further alarm based on the signal and the sensor signal, said computer transmitting the further alarm to the user.

9. The system according to claim 7, further comprising:
a fluid or gas source supplying a fluid or gas to the patient;
a regulator for adjusting a flow rate of the fluid or gas, said regulator being connected to said digital computer;
said computer sending the alarm to said wireless transmitter of said digital computer;
said digital computer sending a signal to said regulator after receiving the alarm; and
said regulator adjusting the flow rate of the fluid or gas after receiving the signal from said digital computer.

10. The system according to claim 7, wherein:
said fluid flow sensor or mass flowmeter of said device is an input mass flowmeter, said input mass flowmeter generating the signal describing a flowrate of oxygen as a function of time;
an oxygen source is connected to an input of said input mass flowmeter;
an oxygen delivery device is connected to an output of said input mass flowmeter;
an output mass flowmeter for measuring flowrate of exhalation sends a signal to said digital computer, the signal describing the flowrate of exhalation as a function of time;
an input of said output mass flowmeter is connected to said oxygen delivery device;
said digital computer transmitting the signal generated by said output mass flowmeter to said server;
said server storing the signal generated by said output mass flowmeter; and
said computer generating an alarm based on the signal generated by the input mass flowmeter and the output mass flowmeter and transmitting the alarm to the user.

11. The system according to claim 8, wherein:
said fluid flow sensor or mass flowmeter is a fluid output sensor generating the signal, the signal being a function of the flowrate of the fluid through said fluid output sensor; and
said sensor is selected from the group consisting of a conductometer, a pH meter, a spectrophotometer.

12. The system according to claim 8, wherein:
said fluid flow sensor or mass flowmeter is an input mass flowmeter or an output mass flowmeter; and
said sensor is selected from the group consisting of a transcutaneous blood oxygen sensor, an oxygen saturation sensor, carbon dioxide sensor, respiratory rate sensor, heart rate sensor, blood pressure sensor, a central venous pressure sensor, and a temperature probe.

13. The system according to claim 8, wherein:
said fluid flow sensor or mass flowmeter is an input mass flowmeter or an output mass flowmeter; and
said sensor is a central venous pressure sensor, including:
an ultrasound probe to be placed over a central blood vessel, said ultrasound probe measuring a deformation and wall motion of the central blood vessel, said ultrasound probe generating a signal that measures the deformation as a function of time,
an inflatable bladder over said ultrasound probe,
a strap for securing the ultrasound probe against skin of the patient overlying the blood vessel; and
a lead connecting said ultrasound probe to said digital computer and carrying the signal from said ultrasound probe to said digital computer; and
said digital computer transmits the signal from said digital computer to said server; and
said computer generates the alarm based on the signal from said input mass flowmeter or said output mass flowmeter and from the signal from said ultrasound probe.

14. The device according to claim 3, wherein said urine collection device includes:
a catheter for collecting the urine from the patient; and
a cannula interconnecting said catheter and said input of said fluid flow sensor or mass flowmeter.

15. The device according to claim 3, wherein said urine collection device includes:
a collection hat for collecting the urine from the patient; and
a cannula interconnecting said collection hat and said input of said fluid flow sensor or mass flowmeter.

16. The device according to claim 3, wherein said urine collection device includes:
a sheet for overlying a toilet bowl, said sheet having a drain formed therein;
an iron disk being disposed on said sheet;
a magnet to be disposed on the toilet bowl; and
a cannula connecting said drain to said input of said fluid flow sensor or mass flowmeter.

17. The device according to claim 3, wherein said urine collection device includes:
a toilet seat with a slot formed therein;
a tray being seated in said slot, said tray having a sheet for covering a toilet bowl, said sheet having a drain formed therein; and
a cannula connecting said drain to said input of said fluid flow sensor or mass flowmeter.

18. A method for measuring glomerular filtration rate, which comprises:
injecting a renally secreted, not absorbed, spectrally-active agent into a bloodstream of a patient;
measuring a flowrate of urine output by the patient;
measuring an absorption of the agent in the urine;
calculating a concentration of the agent as a function of time from the absorption; and
correlating the flowrate as a function of time to the concentration of the agent.

19. The method according to claim 18, which further comprises measuring the flowrate of the urine output with an output fluid flow sensor.

20. The method according to claim 19, which further comprises:
storing the concentration of the dye as a function of time in a server;
storing the flowrate as a function of time in a server;
calculating a glomerular filtration rate from the concentration of the dye as a function of time and the flowrate as a function of time with a computer connected to said server; and
sending an alarm from said computer to a user when said computer detects the glomerular filtration rate falls below a preset minimum.
